(19) United States
(12) Patent Application Publication
(10) Pub. No.: US 2021/0361919 A1
(43) Pub. Date: Nov. 25, 2021

(54) QUICK RELEASE CAPSULES

(71) Applicants: Massachusetts Institute of Technology, Cambridge, MA (US); The Brigham and Women's Hospital, Inc., Boston, MA (US); Novo Nordisk A/S, Bagsværd (DK)

(72) Inventors: Robert S. Langer, Newton, MA (US); Carlo Giovanni Traverso, Newton, MA (US); Alex G. Abramson, St. Louis, MO (US); David Dellal, Boston, MA (US); Christoph Winfried Johannes Steiger, Cambridge, MA (US); Niclas Roxhed, Cambridge, MA (US); Ester Caffarel Salvador, Cambridge, MA (US); Vance Soares, Boston, MA (US); Daniel Minahan, Weymouth, MA (US); Morten Revsgaard Frederiksen, Copenhagen (DK)

(73) Assignees: Massachusetts Institute of Technology, Cambridge, MA (US); The Brigham and Women's Hospital, Inc., Boston, MA (US); Novo Nordisk A/S, Bagsværd (DK)

(21) Appl. No.: 17/055,905

(22) PCT Filed: May 17, 2019
(86) PCT No.: PCT/US2019/032777

§ 371 Date: Nov. 16, 2020

Related U.S. Application Data
(60) Provisional application No. 62/767,710, filed on Nov. 15, 2018.
(60) Provisional application No. 62/672,841, filed on May 17, 2018.

(51) Int. Cl. A61M 31/00; A61M 37/00

(52) U.S. Cl. CPC A61M 31/002; A61M 37/0015; A61M 2037/0023; A61M 2210/1053; A61M 2205/0255; A61M 2205/33; A61M 2205/8281; A61M 2205/0222; A61M 2037/0061

(57) ABSTRACT

Articles for rapid release of components including, for example, quick release capsules, are generally provided. Advantageously, in some embodiments, the articles described herein may be configured to prevent fluid from contacting a component h contained therein (e.g., tissue interfacing component) or payload contained therein until a desired time, e.g., the time at which the component is configured to release from the article to a location internal to a subject (e.g., localize to a tissue wall in the subject). In some embodiments, the article comprises a first compartment and a second compartment not in fluid communication with the first compartment. In some embodiments, the first compartment and second compartment are fluidically isolated. For example, in some cases, the first compartment comprises a mechanism for releasing a component contained within the article and the second compartment comprises the component.

### RELATED APPLICATIONS

This application claims priority under 35 U.S.C. § 119(e) to U.S. Provisional Application Ser. No. 62/672,841, filed May 17, 2018, entitled “QUICK RELEASE CAPSULES,” and to U.S. Provisional Application Ser. No. 62/767,710, filed Nov. 15, 2018, entitled “ACTUATING COMPONENTS AND RELATED METHODS,” each of which are incorporated herein by reference.

### FIELD

The present invention generally relates to articles for rapid release of components including, for example, quick release capsules.

### BACKGROUND

Insulin and other biologic drugs have transformed diabetes from a terminal diagnosis into a manageable chronic illness; however, the need to subcutaneously inject these medicines creates patient discomfort, which in turn delays initiation in treatment regimens and reduces patient compliance. The gastrointestinal (GI) tract, for example, offers an incredible opportunity for diagnosing and treating patients.

Devices may be used to facilitate the delivery of orally ingestible drugs that degrade easily in the GI tract of patients. Macromolecule drugs, a type of active pharmaceutical ingredient (API), in particular may be advantageously included in a device to increase drug bioavailability, because the human body contains enzymes to specifically degrade such APIs. These devices may act by localizing a drug payload next to a tissue wall and allowing the drug to diffuse into the tissue. While devices can be fabricated to deliver to the stomach or buccal space, the small intestine offers a greater amount of vasculature which may facilitate faster drug uptake. In order to reach the small intestine, some drug containing devices protect their payload throughout the upper GI tract and prevent any diffusion of drug out of or enzymes into the device. However, as soon as fluids interact with the drug payload in the device, the drug may diffuse out and begin to degrade.

The development of smart dosage systems and devices to enable this has witnessed significant growth over the preceding decade. Orally ingested drugs generally diffuse through the gastrointestinal tract tissue walls in order to enter the blood stream. Typical ingested pills or devices release their cargo into the gastrointestinal tract randomly such that the cargo (e.g., drug) transits via convection and diffusion to the tissue wall. However, many biologic drugs such as insulin cannot move through the liquid in the GI tract as they may be degraded by enzymes, even if housed in a solid formulation, and/or cannot diffuse readily through the walls of the GI tract.

Accordingly, improved articles and methods are needed.

### SUMMARY

The present invention generally relates to articles for rapid release of components including, for example, quick release capsules.

In one aspect, articles are provided. In some embodiments, an article is configured for administration to a subject. In some embodiments, an article comprises: a capsule having a body comprising a first compartment and a second compartment not in fluid communication with the first compartment, wherein both the first compartment and the second compartment, in a pre-deployment state of the article, are sealed from fluid communication with an environment external to the article; a deployment mechanism associated with the first compartment and configured to eject, from the second compartment, a component for release internally of the subject; a fluidic gate between the first compartment and an environment external to the first compartment, the fluidic gate having a first configuration in which the fluidic gate inhibits fluid communication between the external environment and the first compartment, and a second configuration in which the fluidic gate allows fluid communication between the external environment and the first compartment; and a deployment inhibitor associated with the deployment mechanism, the deployment inhibitor configured to maintain the deployment mechanism in a pre-deployment state until sufficient exposure to a bodily fluid of the subject through a pathway including the fluidic gate in its second configuration, wherein the deployment mechanism is configured to re-configure, in sufficient presence of the bodily fluid, allowing the deployment mechanism to eject the component from the article into an environment internally of the subject.

In some embodiments, an article comprises: a capsule having a body comprising a first compartment and a second compartment, the first compartment comprising a deployment mechanism associated with a fluidic gate embedded in a bottom portion of the first compartment; a plunger disposed within the first compartment and associated with a tissue interfacing component disposed within the second compartment, wherein the plunger is configured to prevent fluidic communication between the first compartment and the second compartment; and a deployment inhibitor associated with the deployment mechanism, the deployment inhibitor configured to maintain the deployment mechanism in a compressed state until exposure to a fluid, wherein the deployment inhibitor is configured to disassociate in the presence of the fluid, releasing the deployment mechanism from compression, wherein the capsule body is sealed.

In some embodiments, the article comprises a capsule, an actuating component disposed within the capsule, the actuating component comprising a central core and three or more arms associated with and extending from the central core, having a first, pre-deployment configuration and a deployed configuration, and at least one arm having a proximal portion and a distal end and a plurality of microneedles disposed near the distal end, the plurality of microneedles comprising an active pharmaceutical agent.

In some embodiments, the plurality of microneedles, at least in the pre-deployment configuration, are oriented external to a geometric center of the capsule.

In some embodiments, the article comprises a capsule, an actuating component disposed within the capsule, the actuating component comprising a central core and three or more arms associated with and extending from the central core, having a first, pre-deployment configuration and a deployed configuration, and at least one arm having a proximal portion and a distal end and a protrusion disposed near the distal end, wherein the actuating component has a pre-deployment configuration within the capsule and a deployed configuration, different than the pre-deployment configuration, external to the capsule.

In some embodiments, the protrusion comprises a plurality of microneedles.

In some embodiments, the article comprises a core, three or more arms associated with and extending from the central core, and a plurality of microneedles disposed proximate a distal end of at least one arm.

In another aspect, methods of administering an active pharmaceutical agent to a subject are provided. In some embodiments, the method comprises administering to the subject a capsule comprising an actuating component disposed within the capsule, the actuating component having a pre-deployment configuration within the capsule, releasing the actuating component, at a location internal to the subject, such that the actuating component obtains a deployed configuration, different than the pre-deployment configuration, wherein the actuating component comprises a core and three of more arms associated with and extending from the central core, and a plurality of microneedles disposed near a distal end of at least one arm, wherein, upon obtaining the deployed configuration, the plurality of microneedles engage with a least a portion of tissue at the location internal to the subject, and exposing the tissue to the active pharmaceutical agent.

In another aspect, methods are provided. In some embodiments, a method for administering a tissue interfacing component to a subject is provided. In some embodiments, the method comprises: administering, to the subject, a capsule having a body comprising a first compartment and a second compartment, a deployment mechanism comprising a deployment inhibitor within the first compartment; exposing the capsule to a fluid having a pH of greater than or equal to 6 such that a fluidic gate having a first configuration and embedded in a bottom portion of the first compartment obtains a second configuration; exposing the deployment inhibitor to the fluid such that the deployment inhibitor disassociates; activating the deployment mechanism such that the deployment mechanism engages the tissue interfacing component disposed within the second compartment; and releasing the tissue interfacing component from the capsule to a location internal to the subject.

Other advantages and novel features of the present invention will become apparent from the following detailed description of various non-limiting embodiments of the invention when considered in conjunction with the accompanying figures. In cases where the present specification and a document Incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic illustration of a self-actuating article, according to one set of embodiments;

FIG. 1B shows two cross-sectional schematic diagrams, each of an exemplary self-actuating capsule, according to one set of embodiments;

FIG. 1C is a schematic diagram of a bottom view of a capsule, according to one set of embodiments;

FIG. 1D is a labeled photograph of a quick release capsule, according to one set of embodiments;

FIG. 2A is an exemplary plot of expulsion forces from a lubricated capsule and an unlubricated capsule, according to one set of embodiments;

FIG. 2B is an exemplary plot of a force profile during expulsion of an unlubricated expanding device, according to one set of embodiments;

FIG. 3A is a photograph of a capsule with a self-orienting system inside, including in the capsule a sucrose-coated spring, in which the sucrose has begun to dissolve in liquid, according to one set of embodiments;

FIG. 3B is a photograph of a capsule with a self-orienting system inside, including a polyethylene glycol (PEG)-coated top cap holding a spring inside the capsule in compression, according to one set of embodiments;

FIG. 4 includes X-ray images and a diagram of a capsule before and after release of an expanding component from the capsule in vivo, which release occurred within 90 minutes, according to one set of embodiments;

FIG. 5 is a schematic diagram of an exemplary timeline of release of an expandable component from a capsule, according to one set of embodiments;

FIG. 6A is a schematic diagram of an exemplary actuating component, according to one set of embodiments;

FIG. 6B is a schematic diagram of an exemplary article comprising an actuating component, according to one set of embodiments;

FIG. 6C is a photograph of an exemplary actuating component, according to one set of embodiments;

FIG. 6D is a photograph of an exemplary plurality of microneedles associated with an actuating component, according to one set of embodiments;

FIG. 6E is a photograph of an exemplary actuating component, according to one set of embodiments;

FIG. 7 is a schematic diagram of a luminal unfolding microinjector (LUMI) (an exemplary actuating component), according to one set of embodiments. Actuating components may be swallowed in enteric capsules and actuate and unfold in the small intestine, injecting drug loaded microneedles into the tissue wall. The microneedle patches and arms may dissolve, for example, within several hours and the dissolved portion(s) of the actuating component pass through the GI tract;

FIGS. 8A-8M shows actuating component fabrication and design specifications, according to one set of embodiments. (A) The actuating component was housed inside of a waterproof chamber until it reached the small intestine. After delivering the actuating component, the capsule broke apart into small pieces and passed through the GI tract. (B) actuating components opened up either in parallel or axially with the small intestine. (C) X-rays confirmed that the capsule actuated and released the actuating component within 2 hours. (D) Unfolded and (E) encapsulated actuating component. (F) Unfolding impact force applied by the arm (n=9, Error Bars=SD). (G) Forces required for arm deflection and (H) torsion (n=9, Error Bars=SD). (I) Percent of devices deployed axially in vivo (n=15). (J) Tissue stretch from unfolding (n=9, Error Bars=SD). (K) Actuating component design space and dependence on torque created from arm length and elastomeric force. (L) Capsule Release time may be depended on molecular weight of polyethylene glycol (PEG) coating. (M) Arm flexural strength before and after dissolution in simulated intestinal fluid at 37° C. The dotted line represented the flexural stress required to break the actuating component arm;

FIGS. 9A-9I show microneedle characterization in the small intestine, according to one set of embodiments. Microneedles comprised polyvinylpyrrolidone. (A) Force and (B) displacement for needle perforation in the small intestine. (C) Microneedles were fabricated using solid active pharmaceutical ingredient (API) powder to increase their drug loading. A single patch 1 cm² held up to 0.3 mg in the tips alone. The microneedle patch pictured contained Texas red dye. (D) Actuating component arms contained an indentation to house insulin loaded microneedles during encapsulation. (E) MicroCT image of a barium sulfate loaded microneedle patch applied to a section of human small intestine using the actuating component. The tissue is outlined in pink. (F) Histology confirmed that needles applied to the small intestine using the actuating component penetrated but did not perforate the tissue. Surgical dye used to coat the needle reached 800 μm below the surface of the tissue. (G) Relative dye transfer over time of microneedles to small intestine tissue. In the control experiment, patches were not penetrated. (H) Texas red microneedle dissolution in human tissue. (I) Optical Coherence Tomography imaging confirmed that microneedles penetrated into the small intestine tissue;

FIGS. 10A-10C show in vivo oral insulin delivery via actuating component (LUMI) in swine, according to one set of embodiments. (A) Blood glucose and (B, C) plasma insulin levels are determined. Two actuating components were deployed in swine jejunum and delivered a total of 0.3 mg of insulin in polyvinylpyrrolidone microneedle (MN) patches. Delivery was compared to an equivalent insulin dose from a microneedle patch dissolved in 0.5 mL sterile saline delivered subcutaneously, a microneedle patch dissolved in 10 mL water delivered to the jejunum, or a microneedle patch applied directly to the jejunum. (n=3, Error Bars=SEM);

FIG. 11 shows impact and static forces generated by the elastomer core in the actuating components, according to one set of embodiments. Two of the arms were fixed in an orientation parallel to the bottom surface. The arm of interest was initially held parallel to the bottom surface, and it was instantaneously released. The arm traveled until it collided with the compression platen. The force applied to the compression platen by the arm was measured over time. The photograph in the lower right corner shows an exemplary actuating component containing a tempered spring steel and mediprene core;

FIG. 12 shows bar and arm shape used for dissolution testing, according to one set of embodiments. Different polyethylene oxide (PEO) and Soluplus® mixtures were evaluated for their dissolution timelines;

FIG. 13 shows photographs of an exemplary actuating component delivered to the small intestine in an enteric capsule, according to one set of embodiments. Stainless steel ball bearings 1 mm in diameter are placed on the arms to aid in visualization. The device is broken up after 2 hours in the small intestine, and the ball bearings begin to pass out of the GI tract within two days;

FIGS. 14A-14C shows penetration characterization for small intestine tissue, according to one set of embodiments. Forces required to displace (A) ex vivo human and (B) in vivo swine small intestine tissue. (C) A comparison between human and swine forces in the small intestine using 32 G needles. (Human tissue: n=10 over 3 small intestines; Swine tissue: n=15 over 3 small intestines. Error bars=SEM.)

FIG. 15 shows exemplary actuating component arms with microneedle patches made with different formulation and active pharmaceutical ingredients, according to one set of embodiments;

FIGS. 16A-16C show exemplary actuating component deployment with hypodermic needle, according to one set of embodiments. (A) Colored MicroCT reconstruction. (B) Needle is same height as microneedles. (C) MicroCT of actuating component deployment. Tissue is outlined in dashed lines.

FIGS. 17A-17B shows optical coherence tomography (OCT) images showing the microneedles mounted in the actuating component arm, according to one set of embodiments. (A) prior insertion and (B) inserted into small intestine after deploying the arm from a 30 degree angle. Arrows point the holes observed in the tissue corresponding to microneedles being inserted;

FIG. 18 shows an in vivo image of swine tissue applied with Texas red loaded microneedle patches, according to one set of embodiments. Patches were applied in vivo. The tissue was harvested and imaged within 3 hours. Each set of patches were applied for varying amounts of time. The control patches were left to sit on top of the tissue, but they were not pressed into the tissue;

FIG. 19 shows optical coherence tomography (OCT) images of microneedles of varying lengths inserted into swine small intestine tissue, according to one set of embodiments. Lighter gray represents small intestine tissue;

FIG. 20 shows OCT images demonstrating dissolution of microneedle patches in ex vivo swine tissue, according to one set of embodiments;

FIG. 21 shows the dissolution of insulin microneedle patches applied to in vivo swine small intestine, according to one set of embodiments. Control patches were laid upon the tissue and all other patches were penetrated into to the tissue; and

FIG. 22 shows an exemplary actuating component fabrication process, according to one set of embodiments. Custom fabricated polydimethylsiloxane (PDMS) mold for creation of actuating component backbone. We used mediprene for the elastomer core and a PEO/Soluplus® mixture for the arms. Metal cores were embedded in the elastomer during heating.

### DETAILED DESCRIPTION

Articles for rapid release of components (e.g., actuating components) including, for example, quick release capsules, are generally provided. Advantageously, in some embodiments, the articles described herein may be configured to prevent fluid from contacting a component contained therein (e.g., tissue interfacing component, actuating component) or payload contained therein until a desired time, e.g., the time at which the component is configured to release from the article to a location internal to a subject (e.g., localize to a tissue wall in the subject). In some embodiments, the article comprises a first compartment and a second compartment not in fluid communication with the first compartment. In some embodiments, the first compartment and second compartment are fluidically isolated. For example, in some cases, the first compartment comprises a mechanism for releasing a component contained within the article and the second compartment comprises the component. Advantageously, keeping the component in a second compartment fluidically isolated from a first compartment may prevent an undesired exposure of the component to a fluid (e.g., gastric fluid) prior to release from the article (e.g., the capsule). In certain embodiments, the article comprises a deployment mechanism (e.g., spring and/or plunger), a deployment inhibitor (e.g., a coating, a sugar coating) associated with the deployment mechanism, and a fluidic gate (e.g., comprising a plug, comprising an enteric plug, comprising an enzymatic plug) associated with the first compartment. In some cases, a component (e.g., a tissue interfacing component, device, expanding device, self-righting device) may be associated with the second compartment.

In some embodiments, the enteric plug comprises an enteric polymer. The term enteric is generally used to describe materials that are stable at relatively highly acidic pH conditions (e.g., pH of less than about 5.5) and susceptible to dissolution at relatively alkaline pH conditions (e.g., pH of between about 6 and about 9). In some embodiments, the enteric polymer includes, but is not limited to, cellulose acetate phthalate (CAP), cellulose acetate trimellitate (CAT), cellulose acetate succinate, hypromellose (INN), hydroxypropyl methylcellulose (HPMC) and derivatives thereof, polyvinyl acetate phthalate, poly(acryloyl-6-aminocaproic acid), e.g., EUDRAGIT® a available from Evonik Industries AG (Essen, Germany)), and/or combinations thereof.

In some embodiments, the fluidic gate comprises an enzymatic plug comprising a polymer configured to be degraded by an enzyme(s). In certain embodiments. The fluidic gate comprises one or more types of materials e.g., an enteric polymer, an enzymatically degradable material, or combinations thereof. For example, in some embodiments, the fluidic gate may comprise a first layer comprising an enteric polymer and a second layer adjacent (e.g., directly adjacent) the first layer comprises a different material (e.g., a different enteric polymer, a material configured to be degraded by an enzyeme(s)).

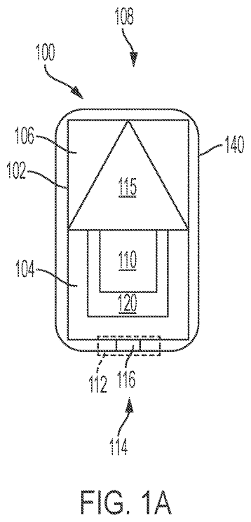

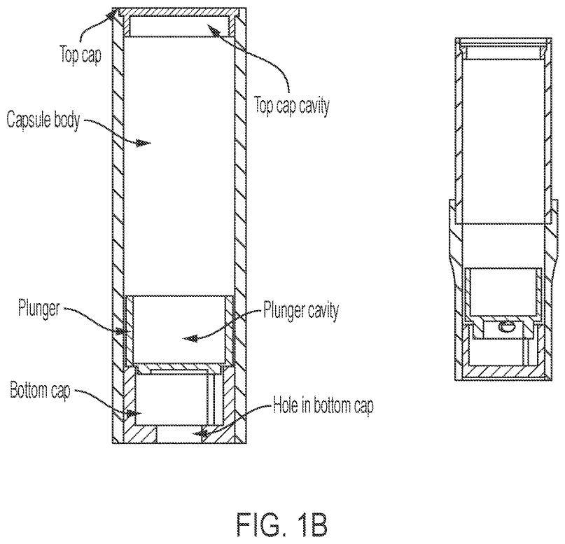

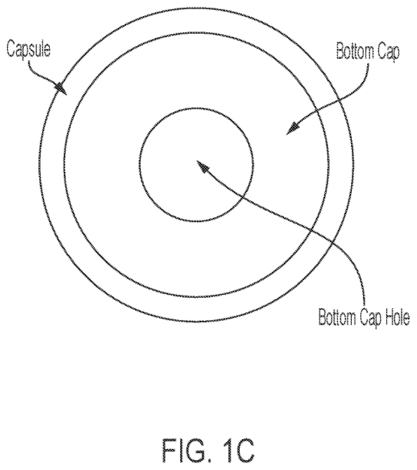

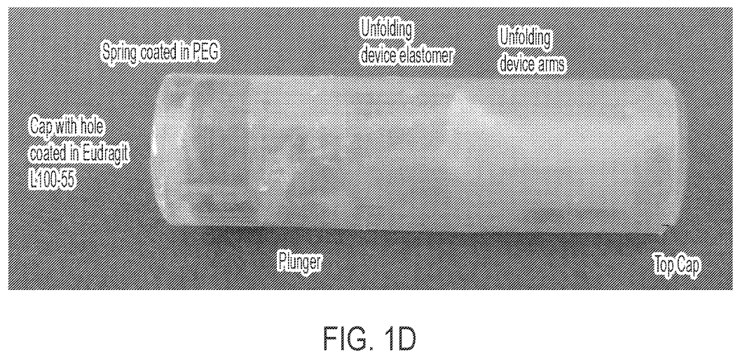

As illustrated in FIG. 1A, in some embodiments, article 100 comprises a capsule 140 having a body 102 comprising a first compartment 104 and a second compartment 106 not in fluid communication with the first compartment 104. In certain embodiments, both first compartment 104 and second compartment 106, in a pre-deployment state of article 100, are sealed from fluid communication with an environment 108 external to the article. In some embodiments, article 100 comprises a deployment mechanism 110 (e.g., spring and/or plunger) associated with (e.g., within) first compartment 104 and configured to eject, from second compartment 106, a component 115 (e.g., tissue interfacing component, device) for release internally of a subject.

In some embodiments, article 100 comprises a fluidic gate 112 (e.g., comprising a plug 116, e.g., comprising an enteric plug) between first compartment 104 and an environment external to the first compartment 114. In some cases, fluidic gate 112 has a first configuration (e.g., in which plug 116 is present) in which fluidic gate 112 inhibits fluid communication between external environment 114 and first compartment 104, and a second configuration (e.g., in which plug 116 is absent and fluidic gate 112 has an unobstructed hole) in which fluidic gate 112 allows fluid communication between external environment 114 and first compartment 104.

In some embodiments, article 100 comprises a deployment inhibitor 120 (e.g., a coating, a sugar coating) associated with (e.g., operably linked with) deployment mechanism 110. In some embodiments, deployment inhibitor 120 is configured to maintain deployment mechanism 110 in a pre-deployment state until sufficient exposure to a bodily fluid of a subject through a pathway including fluidic gate 112 in its second configuration. In some embodiments, deployment mechanism 110 is configured to re-configure, in sufficient presence of a bodily fluid of a subject, allowing deployment mechanism 110 to eject component 115 from article 100 internally of the subject (e.g., to external environment 108).

In some embodiments, an article comprises a capsule having a body comprising a first compartment and a second compartment (e.g., FIG. 1A; FIG. 1B, right). In some embodiments, the capsule body comprises a material non-dissolvable in the fluid. In some embodiments, the capsule body is sealed. In some embodiments, the capsule is made from a non-degradable and impermeable material, such that liquids cannot enter the capsule through the material from which the capsule is made. In some embodiments, the capsule, or at least a portion of the capsule, is press fit e.g., to prevent entry of water into the capsule. In some embodiments, the first compartment and the second compartment are fluidically isolated. In some embodiments, the capsule maintains the component (e.g., tissue interfacing component) in a relatively dehydrated state until release.

The articles described herein may, in some cases, comprise two or more fluidically isolated components. For example, in some embodiments, two or more portions of the article may not be in fluidic communication.

Referring again to FIG. 1A, in some embodiments, first compartment 104 and second compartment 106 are not in fluidic communication. In certain embodiments, deployment mechanism 110 and deployment inhibitor 120 may be fluidically isolated (e.g., not in fluidic communication) with component 115 (e.g., tissue interfacing component). Advantageously, having two or more fluidically isolated components may, in some cases, permit the dissolution and/or actuation of one component (e.g., the deployment mechanism, the deployment inhibitor, the fluidic gate) without dissolution and/or activation of another component (e.g., the tissue interfacing component). By way of example, in an exemplary embodiment, component 115 may be fluidically isolated from deployment mechanism 110 and deployment inhibitor 120 such that, upon exposure of deployment mechanism 110 and/or deployment inhibitor 120 to a fluid, deployment mechanism 110 actuates (e.g., deployment mechanism 110 expands) without exposing component 115 to the fluid. For example, component 115 may comprise an active pharmaceutical ingredient (API) that, upon exposure to the fluid, would at least partially dissolve. Advantageously, preventing exposure of the component to the fluid (e.g., protection of the tissue interfacing component) until a desired time (e.g., after release from the article) may prevent premature dissolution of the API prior to insertion into a tissue of a subject.

In some embodiments, a deployment inhibitor (e.g., a disk or coating associated with a deployment mechanism) is configured such that at least a portion of the deployment mechanism (e.g., spring) is not in fluidic communication with the component (e.g., tissue interfacing component).

In some embodiments, the article comprises one or more caps that can be placed on the top and/or bottom of the capsule. As used herein, the term “cap” may refer to a separate physical piece or an extension of a cylindrical core of a capsule forming or covering a bottom portion or top portion of a capsule. In some embodiments, the article comprises a cap on either end of the capsule. In some embodiments, the capsule comprises a cap associated with the second compartment. In some embodiments, the capsule comprises a cap associated the first compartment.

In some embodiments, the deployment mechanism is associated with a fluidic gate.

In some embodiments, a fluidic gate is embedded in a bottom portion of the first compartment. In some embodiments, the second configuration of the fluidic gate comprises a dissolution of at least a portion of the fluidic gate (e.g., dissolution of a plug, e.g. enteric plug, in the fluidic gate). In some embodiments, the fluidic gate comprises a plug (e.g., an enteric plug). In some embodiments, the fluidic gate comprises an enteric plug. In some embodiments, the cap on an actuation side of the capsule (e.g., on the bottom of the capsule) comprises a fluidic gate, which has a first configuration in which the fluidic gate is sealed (e.g., with a Eudragit® coating) and a second configuration in which the fluidic gate is not sealed, and in its second configuration allows liquid into the capsule.

In certain embodiments, at least a portion of the article may be fluidically isolated from the external environment. For example, referring again to FIG. 1A, in some cases, bottom portion 112 comprises a plug 116. The plug, in certain embodiments, prevents fluid from contacting one or more internal pieces of the article (e.g., component, deployment mechanism, deployment inhibitor) until a desired time and/or location. In some embodiments, the plug may be present in a hole such that the deployment mechanism and deployment inhibitor are not in fluidic communication with the external environment (e.g., until dissolution/removal of the plug).

In some embodiments, the capsule has a particular largest cross-sectional dimension along the transverse axis of the capsule. In some embodiments, the largest cross-sectional dimension along a transverse axis of the capsule is less than or equal to 11 mm, less than or equal to 10 mm, less than or equal to 9 mm, or less than or equal to 8 mm. In certain embodiments, the largest cross-sectional dimension along a transverse axis of the capsule is greater than or equal to 1 mm, greater than or equal to 2 mm, greater than or equal to 4 mm, or greater than or equal to 6 mm. Combinations of the above-referenced ranges are also possible (e.g., less than or equal to 11 mm and greater than or equal to 1 mm, less than or equal to 9 mm and greater than or equal to 1 mm). Other ranges are also possible. In some embodiments, the capsule has a largest cross-sectional dimension along a transverse axis of the capsule of less than or equal to 11 mm.

In some embodiments, the capsule has a particular largest length. In some embodiments, the largest length of the capsule is less than or equal to 26 mm, less than or equal to 24 mm, less than or equal to 22 mm, less than or equal to 20 mm, less than or equal to 18 mm, less than or equal to 16 mm, less than or equal to 15 mm, less than or equal to 14 mm, less than or equal to 12 mm, less than or equal to 10 mm, less than or equal to 9 mm, or less than or equal to 8 mm. In certain embodiments, the largest length of the capsule is greater than or equal to 1 mm, greater than or equal to 2 mm, greater than or equal to 4 mm, or greater than or equal to 6 mm. Combinations of the above-referenced ranges are also possible (e.g., less than or equal to 26 mm and greater than or equal to 1 mm, less than or equal to 15 mm and greater than or equal to 1 mm). Other ranges are also possible. In some embodiments, the capsule has a largest length of less than or equal to 26 mm.

In some embodiments, the first compartment has a particular largest length and the second compartment has a particular largest length such that the sum of the largest length of the first compartment and the largest length of the second compartment is equal to the largest length of the capsule. In some embodiments, the largest length of the first compartment is less than or equal to 99%, less than or equal to 95%, less than or equal to 90%, less than or equal to 80%, less than or equal to 70%, less than or equal to 60%, less than or equal to 50%, less than or equal to 40%, less than or equal to 38%, less than or equal to 36%, less than or equal to 34%, less than or equal to 33%, less than or equal to 32%, or less than or equal to 30% of the largest length of the capsule. In some embodiments, the largest length of the first compartment is at least 1%, at least 2%, at least 4%, at least 6%, at least 8%, at least 10%, at least 12%, at least 14%, at least 16%, at least 18%, at least 19%, at least 20%, at least 22%, at least 24%, at least 26%, at least 28%, at least 30%, at least 40%, at least 50%, at least 60%, at least 70%, at least 80%, at least 90%, or at least 95% of the largest length of the capsule. Combinations of the above-referenced ranges are also possible (e.g., between or equal to 1% and 99% the largest length of the capsule, between or equal to 1% and 40% of the largest length of the capsule, between or equal to 19% and 33% of the largest length of the capsule). Other ranges are also possible.

In some embodiments, the first compartment comprises a deployment mechanism. In some embodiments, the deployment mechanism (e.g., spring and/or plunger) is impermeable to a fluid (e.g., bodily fluid). In certain embodiments, a plunger is impermeable to a fluid. In some embodiments, the deployment mechanism is retained within the capsule body after release. In some embodiments, the capsule further comprises a lubricant associated with the deployment mechanism (e.g., plunger).

In some embodiments, an article comprises a plunger. In some embodiments, a plunger is disposed within the first compartment and associated with a tissue interfacing component disposed within the second compartment. In some embodiments, the plunger is configured to prevent fluidic communication between the first compartment and the second compartment. In some embodiments, the first compartment and the second compartment are separated from one another by a plunger, which prevents liquid from passing from one compartment to another. In certain embodiments, the first compartment and the second compartment are separated from one another by a sealant material (e.g., polyethylene glycol (PEG)), which prevents liquid from passing from one compartment to another. In some embodiments, the sealant material and/or the plunger may be degradable (e.g., under physiological conditions).

In some embodiments, an article comprises a spring (e.g., a coil spring, wave springs, Belleville washers, a beam, a membrane, a material having particular mechanical recovery characteristics). In some embodiments, the spring has a spring constant that provides force for the component (e.g., device) to be pushed out of the capsule. The amount of force provided by the spring may depend on whether or not the component provides pressure on the capsule walls before spring activation. In some embodiments, the first compartment comprises an actuating system comprising one or more holes (e.g., FIG. 1C) coated in Eudragit® polymer, a spring, and a disk made from a brittle material which holds back the spring.

In some embodiments, the spring comprises an elastic material. In certain embodiments, the spring comprises a material selected from the group consisting of nitinol, metals, polymers, stainless steel, spring steel, Ultem PEI resin, polyurethane, polymyte, and combinations thereof.

In certain embodiments, the spring may have a particular spring constant. For example, in some embodiments, the spring constant of the spring is greater than or equal to 0.02 N/mm, greater than or equal to 0.04 N/mm, greater than or equal to 0.05 N/mm, greater than or equal to 0.06 N/mm, greater than or equal to 0.08 N/mm, greater than or equal to 0.1 N/mm, greater than or equal to 0.5 N/mm, greater than or equal to 100 N/m, greater than or equal to 150 N/m, greater than or equal to 200 N/m, greater than or equal to 250 N/m, greater than or equal to 300 N/m, greater than or equal to 350 N/m, greater than or equal to 400 N/m, or greater than or equal to 450 N/m. In certain embodiments, the spring constant of the spring may be less than or equal to 500 N/m, less than or equal to 450 N/m, less than or equal to 400 N/m, less than or equal to 350 N/m, less than or equal to 300 N/m, less than or equal to 250 N/m, less than or equal to 200 N/m, less than or equal to 150 N/m, less than or equal to 100 N/m, less than or equal to 0.5 N/mm, less than or equal to 0.1 N/mm, less than or equal to 0.08 N/mm, less than or equal to 0.06 N/mm, or less than or equal to 0.05 N/mm. Combinations of the above-referenced ranges are also possible (e.g., greater than or equal to 0.02 N/mm and less than or equal to 500 N/m, greater than or equal to 0.02 N/mm and less than or equal to 0.05 N/mm). Other ranges are also possible.

In some embodiments, the spring is compressed by greater than or equal to 1 mm, greater than or equal to 2 mm, greater than or equal to 4 mm, greater than or equal to 6 mm, greater than or equal to 8 mm, greater than or equal to 10 mm, greater than or equal to 15 mm, greater than or equal to 20 mm, or greater than or equal to 25 mm along a longitudinal axis of the spring as compared to the uncompressed length of the spring. In certain embodiments, the spring is compressed by less than or equal to 26 mm, less than or equal to 25 mm, less than or equal to 20 mm, less than or equal to 15 mm, less than or equal to 10 mm, less than or equal to 8 mm, less than or equal to 6 mm, less than or equal to 5 mm, less than or equal to 4 mm, less than or equal to 3 mm, or less than or equal to 2 mm along a longitudinal axis of the spring as compared to the uncompressed length of the spring. Combinations of the above-referenced ranges are also possible (e.g., greater than or equal to 1 mm and less than or equal to 5 mm, greater than or equal to 1 mm and less than or equal to 26 mm). Other ranges are also possible.

In certain embodiments, the spring is configured to release a desirable amount of a stored compressive energy of the spring (e.g., upon exposure of the deployment inhibitor to a fluid such as gastrointestinal fluid). For example, in some embodiments, the spring is configured to release at least 50% of the stored compressive energy of the spring, at least 60% of the stored compressive energy of the spring, at least 70% of the stored compressive energy of the spring, at least 80% of the stored compressive energy of the spring, at least 90% of the stored compressive energy of the spring, at least 92% of the stored compressive energy of the spring, at least 94% of the stored compressive energy of the spring, at least 96% of the stored compressive energy of the spring, at least 98% of the stored compressive energy of the spring, or at least 99% of the stored compressive energy. In certain embodiments, the spring is configured to release less than or equal to 100% of the stored compressive energy of the spring, less than 99% of the stored compressive energy of the spring, less than 98% of the stored compressive energy of the spring, less than 96% of the stored compressive energy of the spring, less than 94% of the stored compressive energy of the spring, less than 92% of the stored compressive energy of the spring, less than 91% of the stored compressive energy of the spring, less than 90% of the stored compressive energy of the spring, less than 80% of the stored compressive energy of the spring, less than 70% of the stored compressive energy of the spring, or less than 60% of the stored compressive energy of the spring. Combinations of the above-referenced ranges are also possible (e.g., at least 50% and less than 98%, at least 92% and less than 98% of the stored compressive energy of the spring, at least 94% and less than 96

In some embodiments, the spring is configured to release the stored compressive energy of the spring within any suitable time of exposing the deployment inhibitor to a fluid (e.g., gastrointestinal fluid).

In certain embodiments, the spring is configured to release the stored compressive energy of the spring (e.g., at least 50% of the stored compressive energy) as described herein within less than 1 min, less than 50 seconds, less than 30 seconds, less than 10 seconds, less than 5 seconds, less than 1 second, less than 100 ms, less than 50 ms, or less than 20 ms of exposing the deployment inhibitor to a fluid. In some embodiments, the spring is configured to release the stored compressive energy of the spring within greater than 10 ms, greater than 20 ms, greater than 50 ms, greater than 100 ms, greater than 1 second, greater than 5 seconds, greater than 10 seconds, greater than 30 seconds, or greater than 50 seconds of exposing the deployment inhibitor to a fluid. Combinations of the above-referenced ranges (e.g., within less than 1 min and greater than 10 ms). Other ranges are also possible.

Any combination of the above-referenced ranges are also possible. For example, in certain embodiments, the spring is configured to release at least 50% of the stored compressive energy of the spring within 1 min of exposing the deployment inhibitor to a fluid. In certain embodiments, the spring is configured to release at least 50% of a stored compressive energy of the spring within 10 sec of exposing the deployment inhibitor to a fluid. In some embodiments, the spring is configured to release less than or equal to 100% of a stored compressive energy of the spring within 1 min of exposing the deployment inhibitor to a fluid. In certain embodiments, the spring is configured to release less than or equal to 100% of the stored compressive energy of the spring within 10 sec of exposing the deployment inhibitor to a fluid.

The spring may have any suitable cross-sectional dimension. In some embodiments, the largest cross-sectional dimension of the (uncompressed) spring is greater than or equal to 1 mm, greater than or equal to 2 mm, greater than or equal to 3 mm, greater than or equal to 4 mm, or greater than or equal to 5 mm. In certain embodiments, the largest cross-sectional dimension of the (uncompressed) spring is less than or equal to 10 mm, less than or equal to 6 mm, less than or equal to 5 mm, less than or equal to 4 mm, less than or equal to 3 mm, or less than or equal to 2 mm. Combinations of the above-referenced ranges are also possible (e.g., greater than or equal to 1 mm and less than or equal to 10 mm). Other ranges are also possible.

In some embodiments, an article comprises a deployment inhibitor (e.g., a disk, a coating, e.g., a sugar coating and/or disk) associated with the deployment mechanism (e.g., a spring and/or plunger). In some embodiments, the deployment inhibitor is configured to maintain the deployment mechanism in a compressed state until exposure to a fluid. In some embodiments, the deployment inhibitor is configured to disassociate in the presence of the fluid, releasing the deployment mechanism from compression.

In certain embodiments, referring again to FIG. 1A, article 100 comprises a deployment inhibitor 120 associated with the deployment mechanism 110. In some cases, the deployment inhibitor 120 is a coating. In some embodiments, the deployment mechanism is at least partially encapsulated within the deployment inhibitor. In some embodiments, deployment inhibitor 120 is a biodegradable coating. In certain embodiments, the coating may have any suitable thickness. For example, the thickness of the coating may be greater than or equal to 3 mm, greater than or equal to 4 mm, or greater than or equal to 5 mm. In certain embodiments, the thickness of the coating may be less than or equal to 6 mm, less than or equal to 5 mm, or less than or equal to 4 mm. Combinations of the above-referenced ranges are also possible (e.g., greater than or equal to 3 mm and less than or equal to 6 mm). In certain embodiments, the biodegradable coating at least partially degrades under physiological conditions. In some cases, the deployment inhibitor may comprise a brittle material. Non-limiting examples of suitable deployment inhibitor materials include sugars (e.g., sucrose) and/or polymers (e.g., polyethylene glycol (PEG), polyvinylpyrrolidone, polyvinylalcohol).

In some embodiments, an article includes a capsule that has a deployment mechanism (e.g., spring) not encapsulated in the deployment inhibitor. In some such embodiments, a deployment inhibitor (e.g., comprising a material which can be degraded under physiological conditions, e.g., sugar, an enteric polymer) is configured as a seal on a top portion of the capsule associated with the second compartment and the component (e.g., device). In some embodiments, a deployment inhibitor configured as a seal on the top portion of the capsule comprises an enteric polymer to ensure that degradation does not occur until the capsule reaches the appropriate location in a patient. In some such embodiments, the deployment mechanism comprises a spring that holds the component (e.g., device) under a constant force until the deployment inhibitor dissolves. Once the deployment inhibitor dissolves, in some embodiments, the spring expands and propels the component (e.g., device) out from the capsule. Non-limiting examples of a deployment inhibitor material include sucrose and PEG (e.g., PEG 3350). In some embodiments wherein the article comprises a spring from the examples provided herein, a coating of greater than or equal to 1 mm and less than or equal to 3 mm in thickness holds the spring in place. In some embodiments in which a deployment inhibitor is configured as a seal on a top portion of the capsule, there is no hole in a bottom portion of the capsule near the first compartment of the capsule.

In some embodiments, the second compartment contains a component (e.g., tissue interfacing component, device) disposed within (e.g., that fits inside of) the compartment. In some embodiments, the component is a tissue interfacing component. In some embodiments, the component is an expanding device. In some embodiments, the component (e.g., tissue interfacing component) is a self-righting article. In some embodiments, the article comprises a capsule that holds an expanding device with an elastomeric core. In some embodiments, the article comprises a capsule that holds a self-orienting device.

In some embodiments, an article is configured for administration to a subject.

In some embodiments, the article is administered to a subject (e.g., orally). In certain embodiments, the article may be administered orally, rectally, vaginally, nasally, or uretherally. In certain embodiments, upon reaching a location internal to the subject (e.g., the gastrointestinal tract), at least a portion of the deployment inhibitor degrades such that the spring extends and/or the tissue interfacing component interfaces (e.g., contacts, penetrates) with a tissue located internal to the subject. In some embodiments, the location internally of the subject is the colon, the duodenum, the ileum, the jejunum, the stomach, or the esophagus. As described above and herein, in some embodiments, a component may be released from the article (e.g., the capsule) such that an active pharmaceutical ingredient is released from the component and/or the component is configured to penetrate of the tissue located internal to the subject.

In some embodiments, a tissue interfacing component is associated with the article (e.g., contained within a compartment of the article prior to release from the article). Non-limiting examples of tissue interfacing components include needles, biopsy punches, microneedles, projectiles, or the like. In certain embodiments, the tissue interfacing component comprises a needle, a biopsy component, a hook, a mucoadhesive patch, or combinations thereof. In some embodiments, the tissue-interfacing component comprises a spring-actuated component. For example, an article comprising a tissue interfacing component (e.g., a needle, a plurality of microneedles) may be administered to a subject such that, the article orients at a location internal of the subject such that the tissue interfacing opponent punctures a tissue proximate the location internal of the subject. In some such amendments, an active pharmaceutical ingredient associated with the article may be released into and/or proximate the tissue.

In some embodiments, the capsule is configured to release the tissue interfacing component within greater than or equal to 1 second, greater than or equal to 2 seconds, greater than or equal to 5 seconds, greater than or equal to 10 seconds, greater than or equal to 30 seconds, greater than or equal to 60 seconds, greater than or equal to 5 minutes, greater than or equal to 10 minutes, greater than or equal to 30 minutes, greater than or equal to 60 minutes, greater than or equal to 2 hours, greater than or equal to 4 hours, greater than or equal to 6 hours, greater than or equal to 12 hours, greater than or equal to 24 hours, or greater than or equal to 36 hours of exposure of the fluidic gate (e.g., enteric plug) to the fluid. In certain embodiments, the capsule is configured to release the tissue interfacing component within less than or equal to 48 hours, less than or equal to 36 hours, less than or equal to 24 hours, less than or equal to 12 hours, less than or equal to 6 hours, less than or equal to 4 hours, less than or equal to 2 hours, less than or equal to 60 minutes, less than or equal to 30 minutes, less than or equal to 10 minutes, less than or equal to 5 minutes, less than or equal to 60 seconds, less than or equal to 30 seconds, less than or equal to 10 seconds, or less than or equal to 5 seconds. Combinations of the above-referenced ranges are also possible (e.g., greater than or equal to 1 second and less than or equal to 48 hours, greater than or equal to 30 seconds and less than or equal to 6 hours). Other ranges are also possible.

In some embodiments, methods are provided. In some embodiments, methods for administering a component (e.g., tissue interfacing component) to a subject are provided.

In some embodiments, a method comprises administering, to the subject, a capsule having a body comprising a first compartment and a second compartment, a deployment mechanism comprising a deployment inhibitor within the first compartment. In some embodiments, a method comprises exposing the capsule to a fluid having a pH of greater than or equal to 5.5 (e.g., greater than or equal to 6) such that a fluidic gate having a first configuration and embedded in a bottom portion of the first compartment obtains a second configuration. In some embodiments, a method comprises exposing the deployment inhibitor to the fluid such that the deployment inhibitor disassociates. In some embodiments, a method comprises activating the deployment mechanism such that the deployment mechanism engages the tissue interfacing component disposed within the second compartment. In some embodiments, a method comprises releasing the tissue interfacing component from the capsule to a location internal to the subject. In some embodiments, a method comprises releasing, from the article, an active pharmaceutical agent during and/or after releasing the tissue interfacing component from the capsule to a location internal to the subject. In some embodiments, a method comprises orienting the article such that a longitudinal axis of the tissue interfacing component is orthogonal to the tissue located proximate to the article.

Those of ordinary skill in the art would understand that the term “spring” is not intended to be limited to coil springs, but generally encompass any reversibly compressive material and/or component which, after releasing an applied compressive force on the material/component, the material/component substantially returns to an uncompressed length of the material/component (e.g., within 40%, within 50%, within 60%, within 70%, within 80%, within 90%, within 95% of the length of the material/component prior to compression).

In certain embodiments, the article comprises an expanding component. Those of ordinary skill in the art would understand the term extending component comprises reversibly and irreversibly compressive materials and are components which, upon stimulating and/or releasing a restraint on the expanding component, the expanding component extends in at least one direction (e.g., along its length). In some embodiments, the expanding component comprises a gaseous composition(s) for expanding the gaseous volume expanding component (e.g., a mixture of baking soda and vinegar, gun powder). I

In some embodiments, the spring and/or expanding component may extend in at least one direction via thermal expansion, swelling (e.g., due to fluid absorption), a gas driven process, a pneumatic process, a hydraulic process, an electrical motor, a magnetic mechanism, a torsional spring mechanism, a chemical gas generator, and/or an explosive reaction. In an exemplary set of embodiments, the spring and/or expanding component may extend in at least one direction upon exposure of the spring and/or expanding component to a fluid (e.g., gastrointestinal fluid).

In some cases, the spring and/or the expanding component may be activated (e.g., extended in at least one direction, returns to an uncompressed length of the component) by any suitable activation mechanism. Non-limiting examples of suitable activation mechanisms include release of a pressure difference, electrical timer, light sensor, color sensor, enzymatic sensor, capacitance, magnetism, activation by applied stress (e.g., shape memory materials), external activation (e.g., applied magnetic field, applied light, reaction with gastrointestinal fluid such as stomach acid), and combinations thereof. In an exemplary set of embodiments, the spring and/or expanding component are activated by interaction (e.g., reaction) with a gastrointestinal fluid.

In some embodiments, the articles described herein may be configured for the deployment of one or more components described in WO2018/213593 entitled “Self-Righting Systems, Methods, and Related Components”, filed on May 17, 2018 which is incorporated herein by reference in its entirety. For example, in some embodiments, the self-righting component comprises a first portion, a second portion adjacent the first portion having a different average density than the first portion, and a hollow portion, wherein the self-righting component is configured and arranged to be encapsulated in an article for rapid release of a component as described herein.

In some embodiments, the self-righting component comprises a first portion, a second portion adjacent the first portion having a different average density than the first portion, and a tissue-interfacing component associated with the self-righting component, wherein a ratio of an average density of the first material to an average density of the second material is greater than or equal to 2.5:1. In some embodiments, the ratio of an average density of the second material to an average density of the first material is greater than or equal to 2.5:1. In some embodiments, the self-righting component is configured to anchor at a location internal to a subject and comprises at least a first portion having an average density greater than 1 g/cm³ wherein a longitudinal axis perpendicular to a tissue-engaging surface of the article is configured to maintain an orientation of 20 degrees or less from vertical when acted on by 0.09*10\hat{ }-4 Nm or less externally applied torque and at least one anchoring mechanism associated with the self-righting component.

In some embodiments, the self-righting component is configured for administration to a location internal to a subject and comprises at least a first portion having an average density greater than 1 g/cm³, the self-righting component has a self-righting time from 90 degrees in water of less than or equal to 0.05 second, at least two tissue interfacing components comprising a tissue-contacting portion configured for contacting tissue, each tissue-contacting portion comprising an electrically-conductive portion configured for electrical communication with tissue and an insulative portion configured to not be in electrical communication with tissue, and a power source in electric communication with the at least two tissue interfacing components.

In some embodiments, the component comprises an outer shell, a spring at least partially encapsulated within the outer shell, a support material associated with the spring such that the support material maintains at least a portion of the spring under at least 5% compressive strain under ambient conditions and a tissue interfacing component associated with the spring.

In some embodiments, the component is configured to anchor at a location internal to a subject and comprises an outer shell, a spring at least partially encapsulated with the outer shell, the spring maintained in an at least partially compressed state by a support material under at least 5% compressive strain, and at least one anchoring mechanism operably linked to the spring.

In some embodiments, the component is configured for administration to at a location internal to a subject and comprises an outer shell, a spring at least partially encapsulated with the outer shell, the spring maintained in an at least partially compressed state by a support material under at least 5% compressive strain, at least two tissue interfacing components comprising a tissue-contacting portion configured for contacting tissue, each tissue-contacting portion comprising an electrically-conductive portion configured for electrical communication with tissue and an insulative portion configured to not be in electrical communication with tissue, and a power source in electric communication with the at least two tissue interfacing components.

### Actuating Components

In some embodiments, the articles described herein may be configured for the deployment of actuating components.

Certain embodiments comprise an actuating component associated with a plurality of protrusions such as (micro)needles (e.g., for administering a therapeutic agent to a subject). In some embodiments, the actuating component may be administered to a subject such that the plurality of microneedles are deployed at a location internal to the subject (e.g., in the gastrointestinal tract). The actuating component may be contained within, in some embodiments, a capsule (e.g., for oral administration to a subject) such as the articles described herein. In some embodiments, the actuating component has a pre-deployment configuration in which the plurality of microneedles have a first orientation and a deployed configuration in which the plurality of microneedles have a second orientation, different than the first orientation.

The articles and actuating components described herein may be useful for, for example, as a general platform for delivery of a wide variety of pharmaceutical agents (e.g., drugs) that otherwise are generally delivered via injection directly into tissue due to degradation in the GI tract. For example, in some embodiments, the actuating components may be configured to deliver pharmaceutical agents at a desired location and/or at a desired time and/or over a desired duration to a subject.

Advantageously, the actuating components described herein may offer several advantages over traditional methods for delivering pharmaceutical agents including, for example, the ability to localize to a surface of tissue located internal to a subject (e.g., tissue in the gastrointestinal tract) and/or allowing loaded pharmaceutical agents to avoid long passage through the gastrointestinal tract before diffusing into the blood stream of a subject. In some embodiments, the actuating components described herein may serve as a platform for delivering pharmaceutical agents that are otherwise susceptible to degradation by enzymes (e.g., in the gastrointestinal tract) to be absorbed at relatively higher bioavailability as compared to traditional administration methods.

The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the article and/or the actuating component.

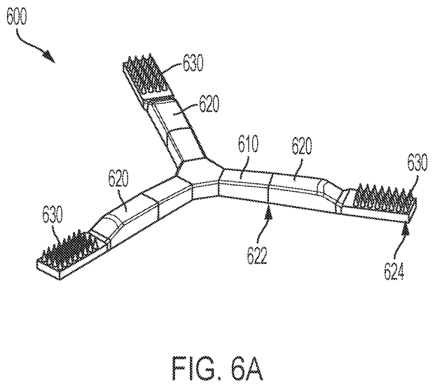

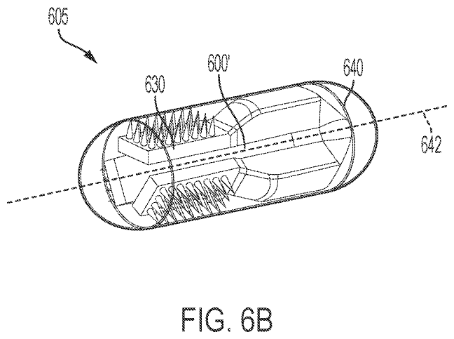

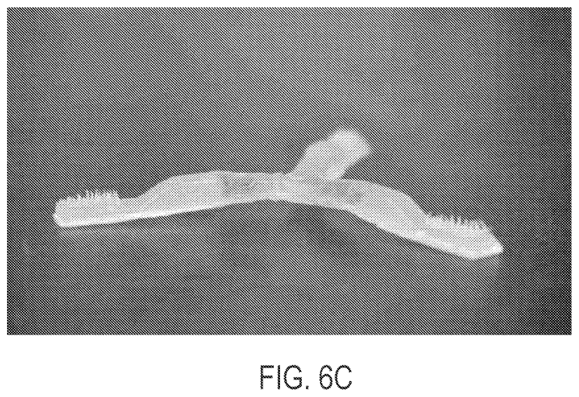

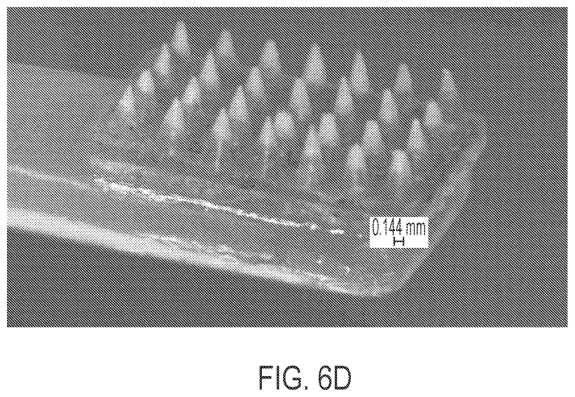

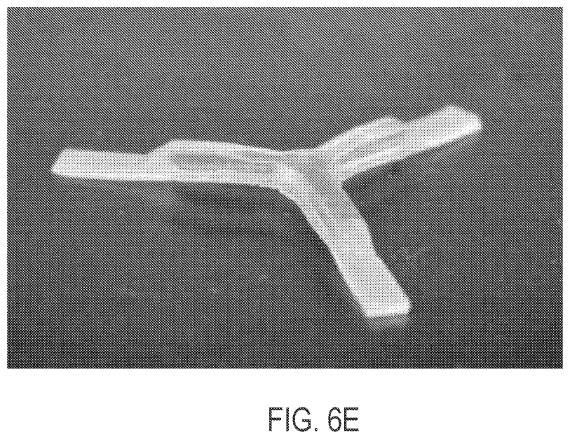

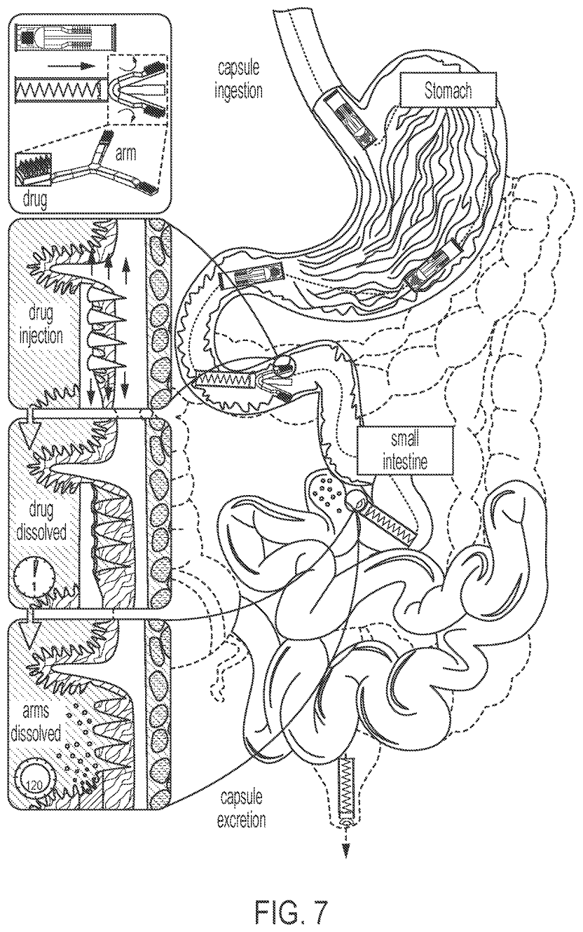

In some embodiments, the actuating component comprises a core, two or more arms associated with the core, and a plurality of microneedles disposed on at least a portion of the arms. For example, as illustrated in FIG. 6A, exemplary actuating component 100 comprises central core 110, arms 120 associated with core 110, and a plurality of microneedles 130 associated with arms 120. While FIG. 6A depicts three arms extended from the central core, those of ordinary skill in the art would understand, based upon the teachings of this specification, that FIG. 6A is meant to be non-limiting and that the actuating component could have 3, 4, 5, 6, 7, 8, 9, 10, or more arms, and each could vary in length, number of protrusions (e.g., (micro)needles), and/or shape.

Additionally, while each arm in FIG. 6A is depicted as having a plurality of microneedles, those of ordinary skill in the art would understand, based upon the teachings of this specification, that not all arms necessarily will be associated with a plurality of microneedles and that each group of microneedles may be the same or different (e.g., same or different loaded pharmaceutical agent, average size, shape, average spacing, and/or average length). One of ordinary skill in the art would also understand, based upon the teachings of this specification, that while much of the disclosure describes the use of a plurality of microneedles, that other protruding features are also possible (e.g., a single needle, a plurality of needles, hooks). In some embodiments, the protruding features are configured to penetrate a surface of layer and/or tissue internal of a subject (e.g., within the gastrointestinal tract).

In some embodiments, as illustrated in FIG. 6A, actuating component 100 is configured such that at least one arm 120 has a proximal portion 122 (relative to the core) and a distal end 124, such that plurality of microneedles 130 are disposed at and/or near distal end 124.

In certain embodiments, the actuating component has a first, pre-deployment configuration (e.g., a folded configuration). For example, as illustrated in FIG. 6B, article 105 comprises a containing structure 140 and actuating component 100 (e.g., as illustrated in FIG. 6A) in a pre-deployment configuration 100′, retained by containing structure 140. In some embodiments, the pre-deployment configuration 100′ comprises at least a portion of plurality of microneedles 130 oriented external to a geometric center 142 of containing structure 140. Advantageously, orientation of the microneedles external to a geometric center of the containing structure permits deployment of the microneedles (e.g., when the actuating component is released from the containing structure and obtains a deployed configuration) such that at least a portion of the microneedles may interface with a surface of tissue located internal to a subject. However, in some embodiments, the microneedles need not be oriented external to a geometric center of the containing structure. For example, in some embodiments, the plurality of microneedles may be oriented at any suitable angle relative to the geometric center of the containing structure such that, upon deployment, the microneedles may engage with a surface at a location internal to a subject.

In some embodiments, the location internally of the subject is the small intestine, the colon, the duodenum, the ileum, the jejunum, the stomach, the rectum, the mouth, or the esophagus. As described above and herein, in some embodiments, a pharmaceutical agent may be released during and/or after penetration of the tissue located internal to the subject by at least a portion of the plurality of microneedles.

While containing structure 140 is depicted as a capsule in FIG. 6B, those of ordinary skill in the art would understand, based upon the teachings of this specification, that FIG. 6B is intended to be non-limiting and other containing structures (e.g., band, surgical thread) are also possible. Based on the application, a capsule may be manufactured to particular specifications or a standard size, including, but not limited to, a 000, 00, 0, 1, 2, 3, 4, and 5, as well as larger veterinary capsules Su07, 7, 10, 12 el, 11, 12, 13, 110 ml, 90 ml, and 36 ml. In some embodiments, the actuating component may be provided in capsules, coated or not. The capsule material may be either hard or soft, and as will be appreciated by those skilled in the art, typically comprises a tasteless, easily administered and/or water soluble compound such as gelatin, starch or a cellulosic material. In some embodiments, the capsule material is not substantially water soluble (e.g., such that the actuating component is protected from external fluid until release from the capsule). In other embodiments, the actuating component is retained in its pre-deployment configuration by a soluble material, such as a band or surgical thread. In some embodiments, the containing structure may be a coating disposed on at least a portion of the actuating component and/or microneedles.

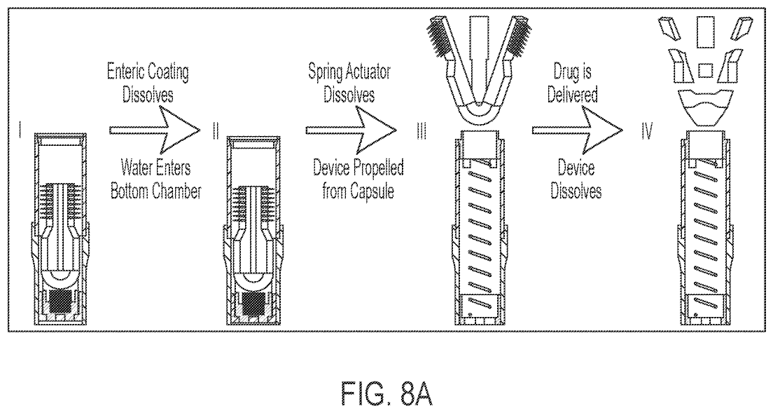

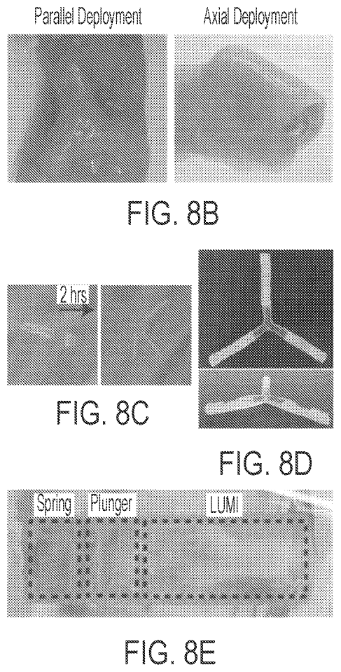

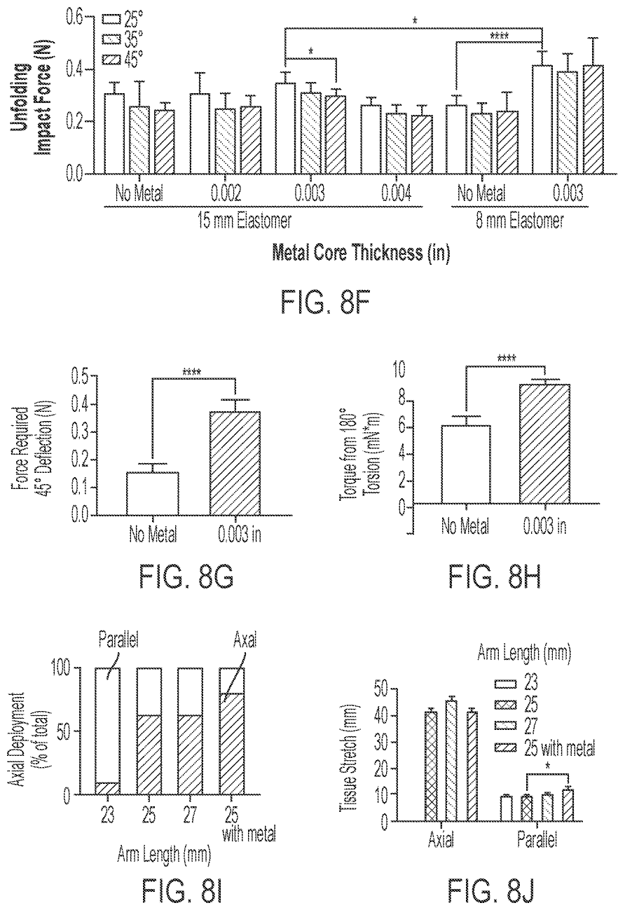

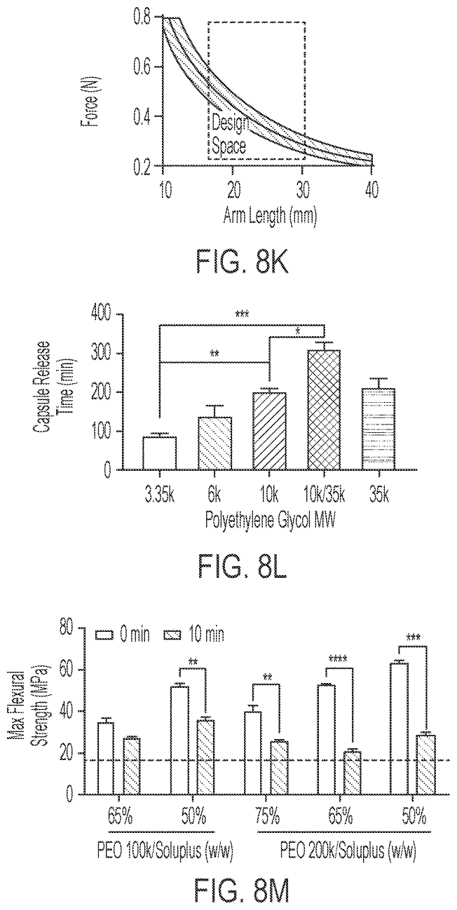

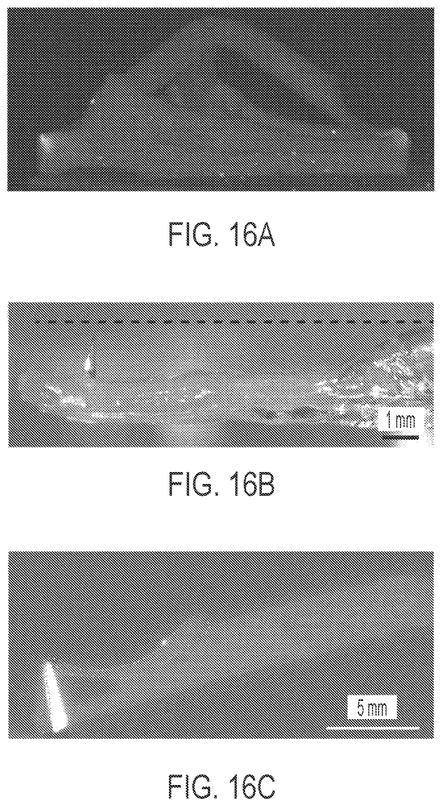

In some embodiments, the actuating component comprises optimal combinations of materials with high and/or low elastic moduli, giving the actuating component the capacity to alter its shape and/or size once the containing structure and/or soluble retaining element is removed. For example, in some embodiments, upon removal of the containing structure (e.g., at least a portion of the containing structure dissolves, degrades, mechanically weakens, and/or mechanically separates such that the actuating component is released), the actuating component obtains a second, deployed configuration, different than the pre-deployment configuration, and external to the containing structure. For example, referring again to FIG. 6A, actuating component 100 is in a deployed configuration (e.g., arms 120 are extended radially from core 110 and such that microneedles 130 are exposed). Again, FIG. 6A is intended to be non-limiting and other deployment configurations are also possible. For example, in some embodiments, the deployment configuration need not necessarily correspond to a fully extended form of the actuating component as illustrated in FIG. 6A. In certain embodiments, the actuating component may have any suitable angle between the arms of the actuating component (see e.g., FIG. 8B and FIG. 16A).

In some cases, the actuating component and/or the article containing the actuating component may be administered to a subject. In some embodiments, the actuating component is administered orally, rectally, vaginally, nasally, or uretherally. In certain embodiments, upon reaching a location internal to the subject (e.g., in the gastrointestinal tract), at least a portion of the containing structure degrades such that the actuating component obtains a deployed configuration and at least a portion of the plurality of microneedles interface (e.g., contacts, penetrates) with the tissue located internal to the subject. For example, in some embodiments, the actuating component has a deployed configuration including a particular size and/or shape in a relaxed state. In certain embodiments, the actuating component may be folded from the deployed configured into a second, pre-deployment configuration. For example, in some cases, the folded/compressed actuating component may be inserted within the capsule or other containment structure in the pre-deployment configuration such that the actuating component can be administered (e.g., orally). The capsule or other containment structure can be, in some cases, configured to dissolve such that the actuating component is released at a particular location internal to the subject whereby upon release, it can reversibly revert to the deployment configuration (e.g., by elastic recoil). In some embodiments, the actuating component is configured to adopt a shape and/or size in vivo that slows or prevents further transit in a body (e.g., gastric, small intestine) cavity until a desired time (e.g., upon dissolution of the microneedles and/or the arms of the actuating component). In some embodiments, the actuating component adopts a shape and/or size configured for temporary retention (e.g., gastric residence) upon release from a capsule/container and/or retaining structure/element. In some embodiments, the actuating component is configured for adopting a shape and/or size configured for gastric deployment after being stored in its encapsulated shape and/or size for durations of less than or equal to 24 hours, less than or equal to 12 hours, less than or equal to 10 hours, less than or equal to 8 hours, less than or equal to 6 hours, less than or equal to 4 hours, less than or equal to 2 hours, less than or equal to 1 hour, less than or equal to 30 minutes, less than or equal to 15 minutes, less than or equal to 10 minutes, less than or equal to 5 minutes, less than or equal to 2 minutes, or less than or equal to 1 minute. In some embodiments, the actuating component is configured for gastric deployment for greater than or equal to 10 seconds, greater than or equal to 30 seconds, greater than or equal to 1 minute, greater than or equal to 2 minutes, greater than or equal to 5 minutes, greater than or equal to 10 minutes, greater than or equal to 15 minutes, greater than or equal to 30 minutes, greater than or equal to 1 hour, greater than or equal to 2 hours, greater than or equal to 4 hours, greater than or equal to 6 hours, greater than or equal to 8 hours, greater than or equal to 10 hours, greater than or equal to 12 hours, or greater than or equal to 18 hours. Combinations of the above-referenced ranges are also possible (e.g., greater than or equal to 10 seconds and less than or equal to 24 hours). Other ranges are also possible. In some embodiments, the actuating component is configured and designed such that a pharmaceutical agent is released from the actuating component (e.g., into a tissue of a subject) for at least a portion of the gastric deployment time. In some embodiments, after deployment, the actuating component is configured to exit the location internal to the subject (e.g., at least a portion of the actuating component degrades, dissolves, mechanically weakens, or mechanically breaks such that the actuating component exits the location internal to the subject).

In some embodiments, a pharmaceutical agent may be administered to a subject by administering an article comprising a containing structure (e.g., capsule) containing an actuating component and releasing the actuating component, at a location internal to the subject, such that the actuating component obtains a deployed configuration, different than the pre-deployment configuration of the actuating component. In some embodiments, upon obtaining the deployed configuration, the plurality of microneedles engage with a least a portion of tissue at the location internal to the subject and the tissue is exposed to the pharmaceutical agent.

In some embodiments, the tissue interfacing component may comprise a plurality of microneedles. In some such embodiments, the plurality of microneedles may have a particular base largest cross-sectional dimension (e.g., diameter of the base), a particular height, and/or a particular spacing.

In some embodiments, the average diameter of the base of the plurality of microneedles is greater than or equal to 100 microns, greater than or equal to 150 microns, greater than or equal to 200 microns, greater than or equal to 250 microns, greater than or equal to 300 microns, greater than or equal to 350 microns, greater than or equal to 400 microns, or greater than or equal to 450 microns. In certain embodiments, the average diameter of the base of the plurality of microneedles is less than or equal to 500 microns, less than or equal to 450 microns, less than or equal to 400 microns, less than or equal to 350 microns, less than or equal to 300 microns, less than or equal to 250 microns, less than or equal to 200 microns, or less than or equal to 150 microns. Combinations of the above-referenced ranges are also possible (e.g., greater than or equal to 100 microns and less than or equal to 500 microns). Other ranges are also possible.

In certain embodiments, the average height of the plurality of microneedles (or protrusion such as a needle) is greater than or equal to 0.1 mm, greater than or equal to 0.2 mm, greater than or equal to 0.5 mm, greater than or equal to 0.7 mm, greater than or equal to 1 mm, greater than or equal to 1.2 mm, greater than or equal to 1.5 mm, greater than or equal to 2 mm, greater than or equal to 3 mm, or greater than or equal to 4 mm. In some embodiments, the average height of the plurality of microneedles/needles is less than or equal to 5 mm, less than or equal to 4 mm, less than or equal to 3 mm, less than or equal to 2.5 mm, less than or equal to 2 mm, less than or equal to 1.5 mm, less than or equal to 1.2 mm, less than or equal to 1 mm, less than or equal to 0.7 mm, less than or equal to 0.5 mm, or less than or equal to 0.2 mm. Combinations of the above-referenced ranges are also possible (e.g., greater than or equal to 0.1 mm and less than or equal to 5 mm). Other ranges are also possible.

In some cases, the average spacing (e.g., spacing between adjacent microneedles in the plurality of microneedles) of the plurality of microneedles may be greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, greater than or equal to 1000 microns, greater than or equal to 1100 microns, greater than or equal to 1200 microns, greater than or equal to 1300 microns, or greater than or equal to 1400 microns. In certain embodiments, the average spacing of the plurality of microneedles is less than or equal to 1500 microns, less than or equal to 1400 microns, less than or equal to 1300 microns, less than or equal to 1200 microns, less than or equal to 1100 microns, less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, or less than or equal to 200 microns. Combinations of the above-referenced ranges are also possible (e.g., greater than or equal to 50 microns and less than or equal to 1500 microns). Other ranges are also possible.

Advantageously, in some embodiments, the plurality of microneedles dissolve relatively quickly (e.g., in less than or equal to 48 hours), reducing and/or eliminating the risk of secondary penetration by the component in undesired locations. In some embodiments, the largest cross-sectional dimension (e.g., length) of the component is designed to be delivered to whichever organ it is targeting to prevent pain and/or undesired perforation of the GI tract.

In some embodiments, the plurality of microneedles comprise a pharmaceutical agent (e.g., API) and a second material (if present), such that the pharmaceutical agent is present in the plurality of microneedles in an amount of greater than or equal to 10 wt % versus the total weight of the plurality of microneedles. In certain embodiments, the pharmaceutical agent is present in the plurality of microneedles in an amount of greater than or equal to 0.1 wt %, greater than or equal to 0.2 wt %, greater than or equal to 0.5 wt %, greater than or equal to 1 wt %, greater than or equal to 2 wt %, greater than or equal to 5 wt %, greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, greater than or equal to 98 wt %, or greater than or equal to 99.1 wt % versus the total weight of the plurality of microneedles. In some embodiments, the pharmaceutical agent is present in the plurality of microneedles in an amount of less than or equal to 100 wt %, less than or equal to 99 wt %, less than or equal to 98 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, less than or equal to 20 wt %, less than or equal to 10 wt %, less than or equal to 5 wt %, less than or equal to 2 wt %, less than or equal to 1 wt %, less than or equal to 0.5 wt %, or less than or equal to 0.2 wt % versus the total weight of the plurality of microneedles. Combinations of the above-referenced ranges are also possible (e.g., greater than or equal to 10 wt % and less than or equal to 100 wt %, greater than or equal to 80 wt % and less than or equal to 100 wt %). Other ranges are also possible.

In some embodiments, the central core of the actuating component comprises the same or different material as the arms of the actuating component. In certain embodiments, the core comprises a spring (e.g., comprising tempered steel and/or nitinol). For example, core may comprises a polymeric material and a spring disposed within the polymeric material.

In some embodiments, the core is configured for undergoing mechanical deformation such that the core does not permanently deform and/or break, and/or is configured to recoil after a particular amount of time such that the actuating component can be selectively retained at a location internally of a subject (e.g., until delivery of the pharmaceutical agent and/or dissolution of the plurality of microneedles and/or arms).

In some embodiments, the core material has particular mechanical properties such that the core material resists brittle breakage but is sufficiently stiff such that it may withstand internal physiological mechanical, chemical, and/or biological challenges to facilitate the ability to maintain residence of the structure or at least the loaded material components of the structure for a desired time interval.

In some embodiments, the actuating component core comprises an elastic polymeric material(s). In certain embodiments, the use of an elastic polymeric material may impart favorable mechanical properties to the structure. For example, in some cases, the core (and/or the actuating component) may be configured for undergoing relatively high compressive forces (e.g., compressive forces present within the stomach and/or intestine of a subject) such that the structure does not break and/or is retained at a location internally of the subject. In certain embodiments, the actuating component and/or core may be configured for being folded (e.g., without breaking). For example, the core may be configured and/or selected for undergoing relatively high levels of bending stresses without breaking and/or without being permanently significantly deformed. In some embodiments, the core and/or the actuating component comprising the core may be configured for substantial recoil. That is to say, after mechanically deforming the core and/or the actuating component comprising the core, the actuating component may return substantially to its original configuration (e.g., the pre-deployment configuration) prior to the mechanical deformation being applied (e.g., the core may be characterized by substantially minimal creep deformation).

Appropriate screening tests may be used to determine suitable materials for use as the core. For example, the core and/or the actuating component may be tested for the capability of undergoing at least about 45 degrees, at least about 60 degrees, at least about 90 degrees, at least about 120 degrees, at least about 150 degrees, or about 180 degrees of mechanical bending deformation without breaking. In certain embodiments, the core and/or the actuating component may be configured for undergoing up to and including about 180 degrees, up to and including about 150 degrees, up to and including about 120 degrees, up to and including about 90 degrees, or up to and including about 60 degrees of mechanical bending deformation without breaking. Any and all closed ranges that have endpoints within any of the above-referenced ranges are also possible (e.g., between about 45 degrees and about 180 degrees, between about 60 degrees and about 180 degrees, between about 60 degrees and about 120 degrees, between about 90 degrees and about 180 degrees). Other ranges are also possible.

In some cases, the core and/or the actuating component may be configured for remaining in a pre-deployment configuration (e.g., at least about 45 degrees of mechanical bending deformation) for a relatively prolonged period of time—for example, in some embodiments, the core has a shelf-life in such a pre-deployment configuration of at least about 24 hours, at least about 1 week, at least about 1 month, at least about 1 year, or at least about 2 years—and still be configured for returning (i.e. recoiling) substantially to its pre-deployment configuration. In certain embodiments, the core has a shelf life in a pre-deployment configuration of up to and including about 3 years, up to and including about 2 years, up to and including about 1 year, up to and including about 1 month, or up to and including about 1 week and be configured for returning (i.e. recoiling) substantially to its deployed configuration. Any and all closed ranges that have endpoints within any of the above-referenced ranged are also possible (e.g., between about 24 hours and about 3 years, between about 1 week and 1 year, between about 1 year and 3 years). Other ranges are also possible.

In some embodiments, the core is relatively flexible. In certain embodiments, the core may be selected such that it is configured for undergoing large angle deformation for relatively long periods of time without undergoing significant non-elastic deformation. In some such embodiments, the core may have a strength of recoil sufficient to substantially return the elastic polymeric component to its deployment configuration within less than or equal to 30 minutes, within less than or equal to 10 minutes, within less than or equal to 5 minutes, within less than or equal to 1 minute, within less than 30 seconds, within less than or equal to 15 seconds, within less than or equal to 10 seconds, within less than or equal to 5 seconds, within less than or equal to 2 seconds, or within less than or equal to 1 second after release of the mechanical deformation (e.g., as applied by the containing structure). In some embodiments, the core may have a strength of recoil sufficient to substantially return the elastic polymeric component to its deployment configuration within greater than or equal to 0.1 seconds, within greater than or equal to 1 second, within greater than or equal to 2 seconds, within greater than or equal to 5 seconds, within greater than or equal to 10 seconds, within greater than or equal to 15 seconds, within greater than or equal to 30 seconds, within greater than or equal to 1 minute, within greater than or equal to 5 minutes, or within greater than or equal to 10 minutes after release of the mechanical deformation. Combinations of the above referenced ranges are possible (e.g., less than or equal to 30 minutes and greater than or equal to 0.1 seconds). Other ranges are also possible.

The core is preferably biocompatible. The term “biocompatible,” as used in reference to a polymeric component, refers to a polymer that does not invoke a substantial adverse reaction (e.g., deleterious immune response) from an organism (e.g., a mammal), a tissue culture or a collection of cells, or invokes only a reaction that does not exceed an acceptable level. In some embodiments, the core comprises polymers, networks of polymers, and/or multi-block combinations of polymer segments, that may comprise polymers or polymer segments that are for example: polyesters—such as including but not limited to, polycaprolactone, poly(propylene fumarate), poly(glycerol sebacate), poly(lactide), poly(glycol acid), poly(lactic-glycolic acid), polybutyrate, and polyhydroxyalkanoate; polyethers—such as including but not limited to, poly(ethylene glycol) and poly(propylene oxide); polysiloxanes—such as including but not limited to, poly(dimethylsiloxane); polyamides—such as including but not limited to, poly(caprolactam); polyolefins—such as including but not limited to, polyethylene; polycarbonates—such as including but not limited to poly(propylene oxide); polyketals; polyvinyl alcohols; polyoxetanes; polyacrylates/methacrylates—such as including but not limited to, poly(methyl methacrylate) and poly(ethyl-vinyl acetate); polyanhydrides; polyvinylpyrrolidone, and polyurethanes. In some embodiments, the polymer is cross-linked. In some embodiments, the core comprises a polymer composite comprising two or more chemically similar polymers or two or more chemically distinct polymers.

According to some embodiments, the actuating component is configured to degrade, dissolve, and/or disassociate into one or more forms capable of passing through a gastrointestinal tract (e.g., after a desired period of time). In some embodiments, the arms of the actuating component may be selected such that each arm dissolves, degrades, mechanically weakens, and/or mechanically separates from the core after a particular residence time period. The term residence time period generally refers to the length of time during which the actuating component described herein is resided at a location internally of a subject as measured from the time initially present in the location internally of the subject to the time at which the device no longer resides at the location internally of the subject due to, for example, degradation, dissolution, and/or exit of at least a portion of the actuating component from the location internally of the subject. In an illustrative embodiment, the actuating component may be orally administered such that the actuating component resides at a location internally of the subject such as the small intestine and exits the small intestine (e.g., after degradation of at least a portion of the actuating component such as the arms), where the residence time period is measured as the length of time between when the actuating component initially resides in the small intestine and when the device exits the small intestine.

In some embodiments, the arms of the actuating component may comprise a degradable material. In some cases, the arms may be configured to mediate disassembly of the actuating component after, for example, delivery of a pharmaceutical agent for the residence time period (e.g., after less than or equal to 48 hours), and safe passage through the lower intestinal tract of the subject. Exit from a location such as the small intestine may be achieved through changes in the mechanical properties of each arm (e.g., via biodegradation) such that the ability to resist passage through the small intestine compromised.

In some embodiments, each arm may have a particular cross-sectional shape. In certain embodiments, the shape may be any suitable cross-sectional shape including circular, oval, triangular, irregular, trapezoidal, square or rectangular, or the like.

In some embodiments, each arm may have a particular length. In some embodiments, the average length of the arms is less than or equal to 30 mm, less than or equal to 28 mm, less than or equal to 26 mm, less than or equal to 25 mm, less than or equal to 20 mm, less than or equal to 15 mm, or less than or equal to 12 mm. In certain embodiments, the average length of the arms is greater than or equal to 10 mm, greater than or equal to 12 mm, greater than or equal to 15 mm, greater than or equal to 20 mm, greater than or equal to 25 mm, greater than or equal to 26 mm, or greater than or equal to 28 mm. Combinations of the above-referenced ranges are also possible (e.g., greater than or equal to 10 mm and less than or equal to 30 mm). Other ranges are also possible.

In some embodiments, each arm may have a particular width. In some embodiments, the average width of the arms is less than or equal to 3.0 mm, less than or equal to 2.8 mm, less than or equal to 2.6 mm, less than or equal to 2.5 mm, less than or equal to 2.0 mm, less than or equal to 1.5 mm, or less than or equal to 1.2 mm. In certain embodiments, the average width of the arms is greater than or equal to 1.0 mm, greater than or equal to 1.2 mm, greater than or equal to 1.5 mm, greater than or equal to 2.0 mm, greater than or equal to 2.5 mm, greater than or equal to 2.6 mm, or greater than or equal to 2.8 mm. Combinations of the above-referenced ranges are also possible (e.g., greater than or equal to 1.0 mm and less than or equal to 3.0 mm). Other ranges are also possible.

The flexural moduli of the arms may be selected to impart desirable features to the actuating component including, for example, the ability to fold and/or bend such that the actuating component can be encapsulated without breaking and/or the ability to withstand compressive forces such as those within the gastric cavity.

In some cases, the actuating component may be configured to deliver a particular amount of pharmaceutical agent per square centimeter of tissue of a subject. For example, in some embodiments, the actuating component is configured to deliver greater than or equal to 0.01 μg, greater than or equal to 0.05 μg, greater than or equal to 0.1 μg, greater than or equal to 0.2 μg, greater than or equal to 0.5 μg, greater than or equal to 0.7 μg, greater than or equal to 1 μg, greater than or equal to 2 μg, greater than or equal to 5 μg, greater than or equal to 10 μg, greater than or equal to 25 μg, greater than or equal to 50 μg, greater than or equal to 100 μg, greater than or equal to 250 μg, greater than or equal to 500 μg, greater than or equal to 1000 μg, or greater than or equal to 2500 μg, greater than or equal to 4000 μg of pharmaceutical agent per square centimeter of tissue of the subject proximate the penetration location of the actuating component. In certain embodiments, the actuating component is configured to deliver less than or equal to 5000 μg, less than or equal to 4000 μg, less than or equal to 2500 μg, less than or equal to 1000 μg, less than or equal to 500 μg, less than or equal to 250 μg, less than or equal to 100 μg, less than or equal to 50 μg, less than or equal to 25 μg, less than or equal to 20 μg, less than or equal to 5 μg, less than or equal to 2 μg, less than or equal to 1 μg, less than or equal to 0.7 μg, less than or equal to 0.5 μg, less than or equal to 0.2 μg, less than or equal to 0.1 μg, or less than or equal to 0.05 μg of pharmaceutical agent per square centimeter of tissue. Combinations of the above-referenced ranges are also possible (e.g., greater than or equal to 1 μg and less than or equal to 5000 m). In some embodiments, the actuating component is configured to deliver greater than or equal to 1 μg and less than or equal to 5000 μg of pharmaceutical agent per square centimeter of tissue of the subject over any suitable time period (e.g., in greater than or equal to 0.1 seconds, in greater than or equal to 0.5 seconds, in greater than or equal to 1 second, in greater than or equal to 5 seconds, in greater than or equal to 30 seconds, greater than or equal to 1 minute, greater than or equal to 5 minutes, 10 minutes, greater than or equal to 30 minutes, greater than or equal to 1 hour, greater than or equal to 4 hours, greater than or equal to 24 hours).

According to some embodiments, the components and methods described herein are compatible with one or more therapeutic, diagnostic, and/or enhancement agents, such as drugs, nutrients, microorganisms, in vivo sensors, and tracers. In some embodiments, the pharmaceutic agent, is a therapeutic, nutraceutical, prophylactic or diagnostic agent. While much of the specification describes the use of pharmaceutical agents, other agents listed herein are also possible.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the active substance is one or more specific pharmaceutical agents. As used herein, the term “pharmaceutical agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the pharmaceutical agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In certain embodiments, the artemisinin derivative is artesunate.

In another embodiment, the pharmaceutical agent is an immunosuppressive agent. Exemplary immunosuppressive agents include glucocorticoids, cytostatics (such as alkylating agents, antimetabolites, and cytotoxic antibodies), antibodies (such as those directed against T-cell recepotors or 11-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In certain embodiments, the pharmaceutical agent is a hormone or derivative thereof. Non-limiting examples of hormones include insulin, growth hormone (e.g., human growth hormone), vasopres sin, melatonin, thyroxine, thyrotropin-releasing hormone, glycoprotein hormones (e.g., luteinzing hormone, follicle-stimulating hormone, thyroid-stimulating hormone), eicosanoids, estrogen, progestin, testosterone, estradiol, cortisol, adrenaline, and other steroids.

In some embodiments, the pharmaceutical agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the pharmaceutical agent is a small molecule drug having molecular weight between 200 Daltons and 400 Daltons, between 400 Daltons and 1000 Daltons, or between 500 Daltons and 2500 Daltons.

In some embodiments, the pharmaceutical agent is selected from the group consisting of active pharmaceutical agents such as insulin, nucleic acids, peptides, bacteriophage, DNA, mRNA, human growth hormone, monoclonal antibodies, adalimumab, epinephrine, GLP-1 Receptor agoinists, semaglutide, liraglutide, dulaglitide, exenatide, factor VIII, small molecule drugs, progrstin, vaccines, subunit vaccines, recombinant vaccines, polysaccharide vaccines, and conjugate vaccines, toxoid vaccines, influenza vaccine, shingles vaccine, prevnar pneumonia vaccine, mmr vaccine, tetanus vaccine, hepatitis vaccine, HIV vaccine Ad4-env Clade C, HIV vaccine Ad4-mGag, dna vaccines, ma vaccines, etanercept, infliximab, filgastrim, glatiramer acetate, rituximab, bevacizumab, any molecule encapsulated in a nanoparticle, epinephrine, lysozyme, glucose-6-phosphate dehydrogenase, other enzymes, certolizumab pegol, ustekinumab, ixekizumab, golimumab, brodalumab, gusellu,ab, secikinumab, omalizumab, tnf-alpha inhibitors, interleukin inhibitors, vedolizumab, octreotide, teriperatide, crispr cas9, insulin glargine, insulin detemir, insulin lispro, insulin aspart, human insulin, antisense oligonucleotides, and ondansetron.

In an exemplary embodiment, the pharmaceutical agent is insulin.

In some embodiments, the tissue-interfacing component described herein comprises two or more types of pharmaceutical agents.

In certain embodiments, the pharmaceutical agent is present in the tissue interfacing component at a concentration such that, upon release from the tissue interfacing component, the pharmaceutical agent elicits a pharmaceutical response.

In some cases, the pharmaceutical agent may be present at a concentration below a minimal concentration generally associated with an active pharmaceutical agent (e.g., at a microdose concentration). For example, in some embodiments, the tissue interfacing component comprises a first pharmaceutical agent (e.g., a steroid) at a relatively low dose (e.g., without wishing to be bound by theory, low doses of pharmaceutical agents such as steroids may mediate a subject's foreign body response(s) (e.g., in response to contact by a tissue interfacing components) at a location internal to a subject). In some embodiments, the concentration of the pharmaceutical agent is a microdose less than or equal to 100 μg and/or 30 nMol. In other embodiments, however, the pharmaceutical agent is not provided in a microdose and is present in one or more amounts listed above.

In some embodiments, between 0.05 wt % to 99 wt % of the pharmaceutical agent initially contained in a plurality of microneedles is released (e.g., in vivo) between 30 minutes and 48 hours. In some embodiments, between about 0.05 wt % and about 99.0 wt % of the pharmaceutical agent is released (e.g., in vivo) from the plurality of microneedles after a certain amount of time. In some embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the pharmaceutical agent associated with the plurality of microneedles is released from the component (e.g., in vivo) within about 48 hours. In certain embodiments, at least about 0.05 wt %, at least about 0.1 wt %, at least about 0.5 wt %, at least about 1 wt %, at least about 5 wt %, at least about 10 wt %, at least about 20 wt %, at least about 50 wt %, at least about 75 wt %, at least about 90 wt %, at least about 95 wt %, or at least about 98 wt % of the pharmaceutical agent associated with the plurality of microneedles is released from the component (e.g., in vivo) within 30 minutes to 24 hours. For example, in some cases, at least about 90 wt % of the pharmaceutical agent associated with the plurality of microneedles is released from the component (e.g., in vivo) within 24 hours.

In certain embodiments, the configuration of the actuating component may be characterized by a largest cross-sectional dimension. In some embodiments, the largest cross-sectional dimension of the pre-deployment (i.e. first) configuration may be at least about 10% less, at least about 20% less, at least about 40% less, at least about 60% less, or at least about 80% less than the largest cross-sectional dimension of the second configuration. In certain embodiments, the largest cross-sectional dimension of the deployed (i.e. second) configuration may be at least about 10% less, at least about 20% less, at least about 40% less, at least about 60% less, or at least about 80% less than the largest cross-sectional dimension of the first configuration. Any and all closed ranges that have endpoints within any of the above referenced ranges are also possible (e.g., between about 10% and about 80%, between about 10% and about 40%, between about 20% and about 60%, between about 40% and about 80%). Other ranges are also possible.

In some embodiments, the configuration of the actuating component may be characterized by a convex hull volume of the actuating component. The term convex hull volume is known in the art and generally refers to a set of surfaces defined by the periphery of a 3-D object such that the surfaces define a particular volume. In some embodiments, the convex hull volume of the first configuration may be at least about 10% less, at least about 20% less, at least about 40% less, at least about 60% less, or at least about 80% less than the convex hull volume of the second configuration. In certain embodiments, the convex hull volume of the second configuration may be at least about 10% less, at least about 20% less, at least about 40% less, at least about 60% less, or at least about 80% less than the convex hull volume of the first configuration. Any and all closed ranges that have endpoints within any of the above referenced ranges are also possible (e.g., between about 10% and about 80%, between about 10% and about 40%, between about 20% and about 60%, between about 40% and about 80%). Other ranges are also possible.

Those skilled in the art would understand that the differences between the first configuration and the second configuration do not refer to a swelling or a shrinking of the actuating component (e.g., in the presence of a solvent), but instead refers to a change in shape and/or orientation of at least a portion of the actuating component (e.g., in the presence of a stimulus such as heat and/or mechanical pressure/compression), although some degree of swelling or shrinking may occur between the two configurations.

In some embodiments, the second configuration is constructed and arranged such that the actuating component is retained at a location internal of a subject, and the first configuration is constructed and arranged such that the actuating component may be encapsulated (e.g., for oral delivery of the actuating component within a capsule). In some cases, the second configuration is sufficiently large such that the actuating component is retained at a location internal of the subject and the first configuration is sufficiently small such that the actuating component may fit within a particular size capsule suitable for oral delivery to a subject.

In certain embodiments, the actuating component may be polymerized and/or cast in a deployment configuration, mechanically deformed such that the actuating component obtains a pre-deployment configuration, and placed in a capsule or restrained by some other containment component. The actuating component may be mechanically deformed using any suitable method including, for example, bending, twisting, folding, molding (e.g., pressing the material into a mold having a new shape), expanding (e.g., applying a tensile force to the material), compressing, and/or wrinkling the actuating component. The actuating component may maintain the pre-deployment configuration for any suitable duration prior to stimulation/release, as described herein. Advantageously, certain embodiments of the actuating components described herein may be relatively stable in the deployed and/or pre-deployment configurations such that the actuating component may be stored for long periods of time without significant degradation of mechanical properties of the core, arms, and/or microneedles. In some embodiments, the actuating component may be stable under ambient conditions (e.g., room temperature, atmospheric pressure and relative humidity) and/or physiological conditions (e.g., at or about 37° C., in physiologic fluids) for at least about 1 day, at least about 3 days, at least about 7 days, at least about 2 weeks, at least about 1 month, at least about 2 months, at least about 6 months, at least about 1 year, or at least about 2 years. In certain embodiments, the actuating component has a shelf life of less than or equal to about 3 years, less than or equal to about 2 years, less than or equal to about 1 year, less than or equal to about 1 month, less than or equal to about 1 week, or less than or equal to about 3 days. Any and all closed ranges that have endpoints within any of the above-referenced ranged are also possible (e.g., between about 24 hours and about 3 years, between about 1 week and 1 year, between about 1 year and 3 years). Other ranges are also possible.

In some embodiments, the actuating component in the pre-deployment configuration may recoil such that the actuating component reverts to the deployed configuration. For example, in some embodiments, the actuating component in the pre-deployment configuration is contained within a capsule and delivered orally to a subject. In some such embodiments, the actuating component may travel to the stomach and the capsule may release the actuating component from the capsule, upon which the actuating component obtains (e.g., recoils to) the deployed configuration (e.g., in the absence of forces applied by the capsule or other containment structure).

As described herein, in some embodiments, the core, arms, and/or microneedles of the actuating component may be cast, molded, and/or cut to have a particular shape, size, and/or volume. In some embodiments, the core, arms, and/or microneedles are adhered via an adhesive. In certain embodiments, the core, arms, and/or microneedles are heated such that the core, arms, and/or microneedles are coupled (e.g., via bonding and/or entanglement). In some embodiments, the microneedles may be arranged such that a major axis of each microneedle is substantially perpendicular to a major plane of each arm. In some embodiments, the micorneedles may be arranged such that the major axis of each microneedle is oriented at an angle of greater than or equal to 45 degrees and less than or equal to 90 degrees relative to a major plane of each arm.

In certain embodiments, the arms are arranged based on bio-inspired flower bud designs in which a number (N) of radial spokes or petals project from a central linking core. In some embodiments, these radial projections each have an internal sector angle of approximately 360°/N, where N is the total number of radial projections. In some cases, this enhances the packing volume of the encapsulated structure, thus increasing drug carrying capacity. In some embodiments, the arms are formed of a material with a relatively high elastic modulus to increase the resistance to compression and duration of gastric residence, as described herein.

Any terms as used herein related to shape, orientation, alignment, and/or geometric relationship of or between, for example, one or more articles, compositions, structures, materials and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elipitical/elipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described.

As used herein, the terms “oligomer” and “polymers” each refer to a compound of a repeating monomeric subunit. Generally speaking, an “oligomer” contains fewer monomeric units than a “polymer.” Those of skill in the art will appreciate that whether a particular compound is designated an oligomer or polymer is dependent on both the identity of the compound and the context in which it is used.

One of ordinary skill will appreciate that many oligomeric and polymeric compounds are composed of a plurality of compounds having differing numbers of monomers. Such mixtures are often designated by the average molecular weight of the oligomeric or polymeric compounds in the mixture. As used herein, the use of the singular “compound” in reference to an oligomeric or polymeric compound includes such mixtures.

As used herein, reference to any oligomeric or polymeric material without further modifiers includes said oligomeric or polymeric material having any average molecular weight. For instance, the terms “polyethylene glycol” and “polypropylene glycol,” when used without further modifiers, includes polyethylene glycols and polypropylene glycols of any average molecular weight.

As used herein, a “fluid” is given its ordinary meaning, i.e., a liquid or a gas. A fluid cannot maintain a defined shape and will flow during an observable time frame to fill the container in which it is put. Thus, the fluid may have any suitable viscosity that permits flow. If two or more fluids are present, each fluid may be independently selected among essentially any fluids (liquids, gases, and the like) by those of ordinary skill in the art. In some embodiments, the fluid is a gastric fluid (e.g., in some cases may comprise a gel like mucus and/or small food particles).

### EXAMPLES

The following examples are intended to illustrate certain embodiments described herein, including certain aspects of the present invention, but do not exemplify the full scope of the invention.

### Example 1

A spring mechanism (an example of a deployment mechanism) could fit inside of a bottom cap of a capsule (e.g., FIG. 1B). A component (e.g., a device) could fit inside of the capsule body. The left schematic diagram in FIG. 1B shows a capsule where the body comprises one compartment, while the right schematic diagram shows a capsule body comprising a first compartment and the second compartment. In some embodiments, a capsule (e.g., represented by the right schematic diagram in FIG. 1B) can break up into pieces that are at a maximum 9 mm×15 mm in size. In some embodiments, the capsule is made completely of bioinert, biocompatible, and/or biodegradable ingredients.

A hole at the bottom of the capsule (e.g., FIG. 1C) could be coated with an enteric polymer (an example of a fluidic gate in its first configuration). Upon removal of the enteric polymer, e.g., by dissolving the enteric polymer in liquid, the hole could allow liquid to enter and release a deployment mechanism (e.g., spring) from a deployment inhibitor (e.g., a sugar coating).

In some embodiments, a component (e.g., an unfolding device) is located inside of a capsule (e.g., FIG. 1D). A portion of the component (e.g., device) could sit flush against the edges of a capsule close to an elastomer, and a second portion of the component may not touch the capsule walls. This configuration may facilitate a pressure sensitive payload attached to the second portion of the component. A spring (an example of a deployment mechanism) could be compressed and held in place by a PEG 3350 coating. There could be a plunger located in between a spring and a component (e.g., device) to create a watertight seal.

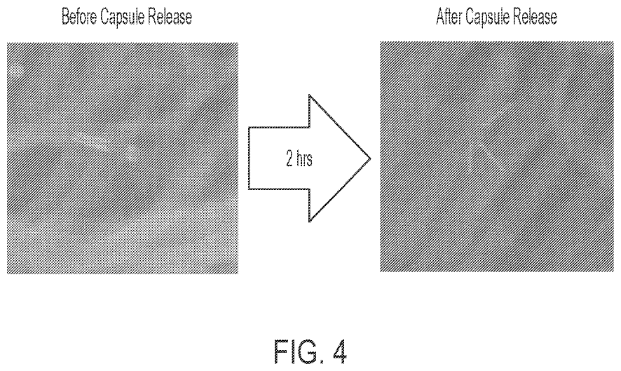

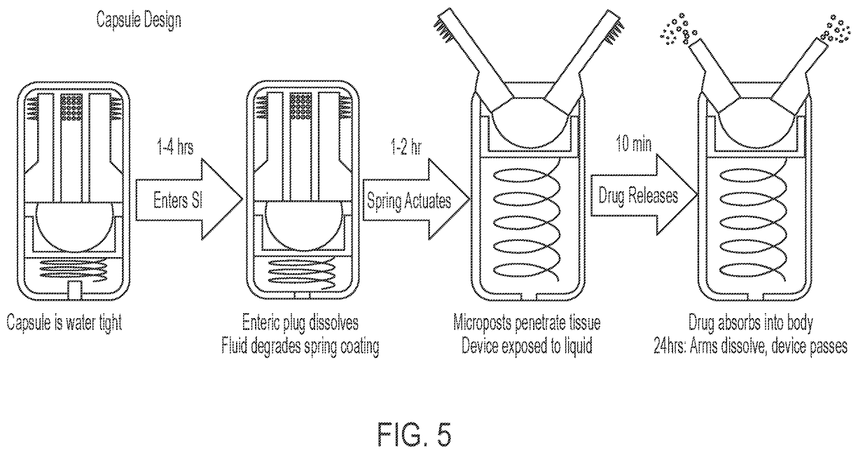

A non-limiting example of a device that can fit inside a second compartment of a capsule body is an expanding device (e.g., in FIG. 4, FIG. 5). In some embodiments in which a capsule containing a component (e.g., an expanding device) is ingested by a patient, e.g., for a consistent drug delivery regimen, the dimensions of a capsule are 9 mm in diameter (an example of a largest cross-sectional dimension along a transverse axis of the capsule) by 15 mm in length (an example of a largest length), e.g., to minimize the obstruction rate. These dimensions have been approved by the Food and Drug Administration (FDA) for daily dosed non-degradable devices. In some embodiments with the capsule having dimensions 9 mm in diameter by 15 mm in length, a first compartment in the capsule (e.g., in which a spring is located) is 5 mm in length and a second compartment in the capsule (e.g., in which a component such as a device is located) is 10 mm in length. In some embodiments in which a capsule is prescribed for infrequent use, such as during a doctor's visit, the dimensions of a capsule are 11 mm in diameter by 26 mm in length (or 9 mm by 15 mm). In such embodiments, a first compartment in the capsule (e.g., in which a spring is located) is between or equal to 5 mm and 8 mm in length, and a second compartment in the capsule (e.g., in which a component such as a device is located) is between or equal to 18 mm and 21 mm in length.

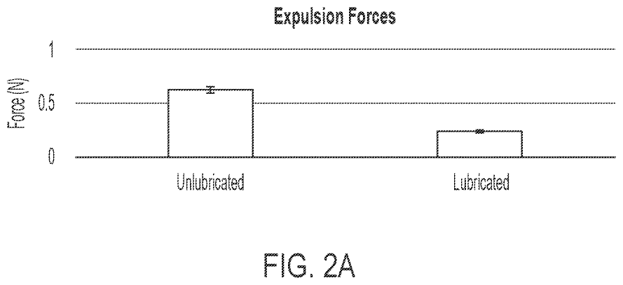

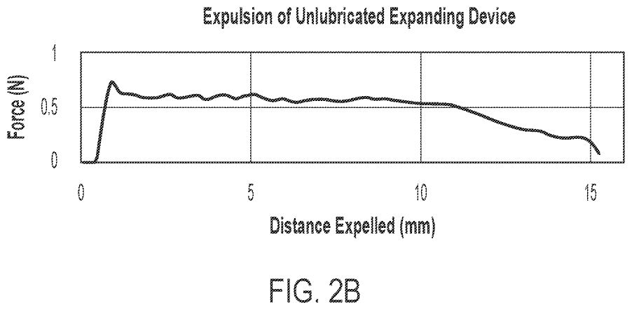

In some embodiments, a portion of a component (e.g., device) inside a capsule is placed flush against a capsule wall. In such embodiments, due to the force applied by the component (e.g., comprising an elastomer) on the capsule wall, there is greater friction to be overcome in order to eject the component out of the capsule, relative to a component without a portion placed flush against a capsule wall. Force calculations that have been conducted show this ejection force could be around 0.6 N for non-lubricated systems. To minimize this force, in some embodiments, the article comprises a lubricant (e.g., an oil, a PAM cooking spray product), which could reduce the ejection force by approximately 0.4 N (e.g., FIG. 2A). In some embodiments, a lubricated capsule results in only approximately 33% of the force required to eject a component (e.g., expanding device) from an unlubricated capsule counterpart (e.g., FIG. 2A). In some embodiments, based on tests to characterize force necessary to eject a component (e.g., device) from a capsule (e.g., FIG. 2B), a spring (an example of a deployment mechanism) exerts more than 0.2N of force on a lubricated device when the spring is compressed to 75% of the length of the capsule. In some embodiments, the force to overcome friction in a capsule during expulsion of an unlubricated component does not decline significantly during the course of an expulsion event (e.g., FIG. 2B). In some embodiments, the free length of a spring is at least 80% of the total length of a capsule in which it resides, or longer, to ensure that a component (e.g., device) is ejected from the capsule.

In some embodiments, a deployment mechanism is a spring that is held in a compressed state by encapsulation inside of a degradable material (e.g., PEG 3350, sucrose). In some embodiments, the spring has a spring constant of greater than or equal to 0.02 N/mm and less than or equal to 0.05 N/mm, or greater than or equal to 0.05 N/mm, in order to ensure that it pushes with enough force to release a component (e.g., device) from the capsule. In some embodiments, a spring has a free length of greater than or equal to 20.8 mm and less than or equal to 35 mm. In some embodiments, the spring has a shorter solid length of greater than or equal to 1.5 mm and less than or equal to 8 mm. In some embodiments, the outer diameter of the spring is close to the inner diameter of the capsule in order to ensure that the spring expands without kinking. In some embodiments, the spring sits inside of a bottom cap of a capsule. In some embodiments, a bottom cap lip and the capsule each have a wall thickness of approximately 0.4 mm, the capsule has an inner radius of 3.4 mm, and a spring inside the capsule has a radius of less than or equal to 2.7 mm. In some embodiments, a spring with a radius of less than or equal to 2.7 mm takes into account a 0.4 mm wall thickness of a cap lip as well as a 0.15 mm tolerance between the cap lip and the inner diameter of the capsule as well as the spring of the cap lip. In some embodiments, the spring is made from a biocompatible material, such as stainless steel.

In some embodiments, liquid used to dissolve a deployment inhibitor (e.g., sugar) enters a capsule through a hole in a bottom cap. In some embodiments, this hole is as large as possible to let in liquid, but not so large as to allow the deployment mechanism (e.g., spring) to pass through the hole. In some embodiments, the hole is not so large as to compromise the integrity of the device. In certain embodiments, the hole is 2.9 mm in diameter. The hole diameter can range in size from greater than or equal to 1 mm and less than the spring diameter (e.g., FIG. 1C). In some embodiments, the article comprises multiple holes. In certain embodiments, the article comprises a single large hole rather than a plurality of smaller holes, which single hole advantageously allows liquid to enter the article more easily. For example, if the surface of the bottom of the article is hydrophobic, water may be prevented from entering small holes in the bottom of the article. In some embodiments, the fluidic gate in the bottom of the capsule includes a hole (covered or blocked in a first configuration, free in a second configuration) having the same diameter as the inner diameter of the capsule. In some embodiments, the hole is 150 microns smaller than the inner diameter of the capsule to allow for tolerance. The hole may have any suitable shape (e.g., square, rectangle, oval).

In some embodiments, to ensure that the deployment mechanism is not freed from the capsule body, which would increase the risk of perforating the tissue wall, the capsule comprises a stopping mechanism. A non-limiting example of a stopping mechanism is one or more strings adhered to the bottom of the capsule and to the deployment mechanism (e.g., spring, plunger). In some embodiments, the strings are made from a biocompatible material and are strong enough to counteract the force exerted by the deployment mechanism (e.g., spring). In some embodiments, the string is at most as long as the capsule so that the spring is never exposed to the surrounding tissue. Another non-limiting example of a stopping mechanism comprises a plurality of posts (e.g., that protrude from the sides of the capsule walls) which contact and/or restrict the deployment mechanism.

In some embodiments, the deployment mechanism comprises a plunger. In some embodiments, the diameter of the plunger is configured to propel a component (e.g., device) out from the capsule is equal in length to the internal diameter of the capsule. In certain embodiments, the diameter of the plunger is 250 microns smaller than the inner diameter of the capsule. In some embodiments, this tolerance of 250 microns minimizes friction and keeps the second compartment watertight. In some embodiments, without wishing to be bound by theory, according to Saint-Venant's principle, to prevent the plunger from rotating as it moves through the capsule, the height of the plunger is at least three times greater than the diameter of the capsule in a high friction environment, 1.6 times greater than the capsule diameter in a low friction environment, and always at least equal to the capsule diameter. In some embodiments, to create more space for a component (e.g., device), the plunger includes a cavity.

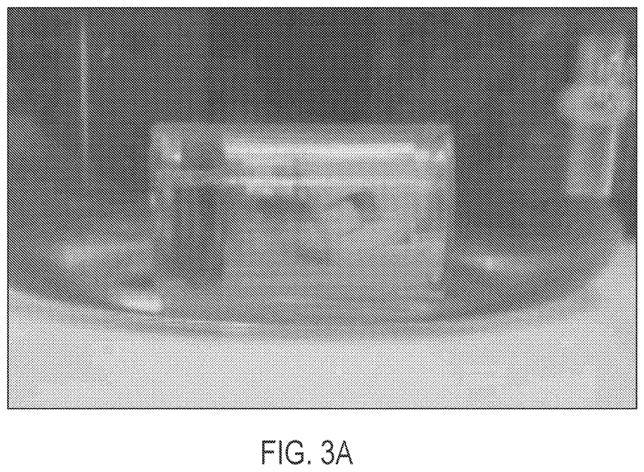

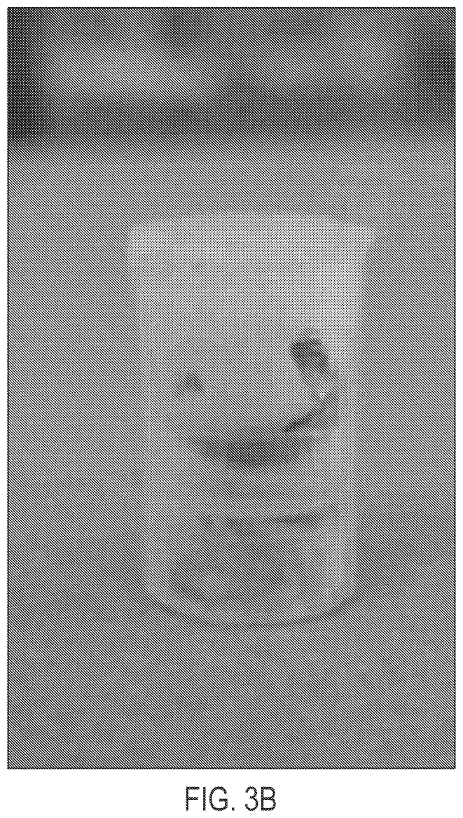

Another non-limiting example of a component (e.g., device) that may be contained inside of a second compartment in a capsule body is a self-righting system (e.g., FIG. 3A). In some embodiments, the component (e.g., device) has a diameter of 8.5 mm so that it can fit inside a 00 capsule. In some such embodiments, the inner diameter of a capsule is at least 8.5 mm in length, and in some cases at least 200 microns longer than 8.5 mm in length. In some embodiments, the component (e.g., device) is 11 mm in height. In some such embodiments, the second compartment is 11 mm in length, and in some cases at least 300 microns longer. In some embodiments, the length of the capsule is greater than or equal to 15 mm. In some such embodiments, the free length of the spring inside the capsule is at least 75% of the length of the capsule. In some embodiments, a self-righting system is not in contact with the interior wall of the capsule and the distance traveled by the spring is smaller to eject the self-righting system rather than an expanding device, and therefore there is less friction in expelling the self-righting system from a capsule then expelling an expanding device from a capsule. In some such embodiments, the force exerted by the spring is greater than or equal to the gravitational force of a self-righting system (e.g., 0.01 N), and in some cases at least 25% larger than the gravitational force of the self-righting system. In some embodiments, a capsule containing a self-righting system also has a stopping mechanism to ensure that a spring in the capsule is not exposed to an environment exterior to the capsule, which would otherwise increase the risk of tissue perforation. In some embodiments, a capsule containing a self-righting system has the same tolerances as those for a capsule containing an expanding device.

In some embodiments, bodily fluid (or a test fluid, e.g., water) passes through a hole in a bottom cap of a capsule, dissolving a sugar (e.g., sucrose) coating on a spring inside a first compartment of the capsule (e.g., FIG. 3A). In some embodiments, this hole is coated with an enteric polymer (an example of a portion of a fluidic gate to form a first configuration of the fluidic gate) such that the sugar only dissolves in the small intestine. In some embodiments, once the sugar begins to dissolve, the spring releases and sends a component (e.g., device) out of the capsule.

Another non-limiting configuration of a capsule includes a spring not encapsulated in a degradable material. In some such embodiments, a degradable material is configured as a seal on a cap of the capsule closest to the component (e.g., device) (e.g., FIG. 3B). In some embodiments, the degradable material configured as a seal is coated with an enteric polymer to ensure that degradation does not occur until the capsule reaches the appropriate location in a patient. In some such embodiments, the spring holds the component (e.g., device) under a constant force until the cap coating dissolves. Once the cap coating dissolves, in some embodiments, the spring expands and propels the component (e.g., device) out from the capsule. Non-limiting examples of a cap coating include sucrose, PEG (e.g., PEG 3350) enteric polymers (e.g., Eudragit L100-55). In some embodiments wherein the article comprises a spring from the examples provided herein, a coating of greater than or equal to 1 mm and less than or equal to 3 mm in thickness holds the spring in place. In some embodiments, a coating is located at a cap lip. In some embodiments in which a degradable material is configured as a seal on a top cap near the second compartment of the article, there is no hole in the bottom cap near the first compartment of the article.

In some embodiments, a capsule includes a PEG-coated top cap (e.g., FIG. 3B). In some embodiments, the PEG is coated with an enteric polymer which dissolves only in the small intestine. In some embodiments, once the PEG begins to dissolve, the spring force overpowers the PEG coating and pops the cap off, releasing the component (e.g., device).

### Example 2—Device Design

The following example describes the design and characterization of an exemplary actuating component (e.g., a luminal unfolding microinjector (LUMI)) and related articles. The actuating component in this example generally utilized the tube like geometry of the small intestine to create multiple points of contact with the tissue (FIGS. 8A-8E). Initially swallowed in a custom designed enteric capsule, the device employed an elastomeric core to quickly unfold and expand within the gastrointestinal (GI) tract. Each of the device's three degradable arms propelled a dissolving drug loaded microneedle patch into the tissue wall. These arms stretched the tissue in multiple directions and allowed the tissue to exert an opposing force on the microneedles. We optimized the force from the elastomer to ensure maximal needle insertion while avoiding perforation. The elastomeric core and the arm geometry maximized both the safety and efficacy of the system.

When exiting the capsule, the exemplary actuating component opened in one of two orientations: either in a plane parallel or perpendicular to the central axis of the small intestine (FIG. 8B). In either orientation, the microneedles made contact with the tissue wall; however, the perpendicular deployment, hereinafter referred to as axial deployment, led to a greater stretch. A geometric analysis of the opening event demonstrated that an actuating component arm's length greater than it/3 times the diameter of the small intestine was able to stretch the tissue during any possible opening configuration.

Varying the arm length and unfolding angle generally affected the amount of force delivered by the actuating component core (FIG. 8F and FIG. 11). Devices with longer arms demonstrated less angular expansion before making contact with tissue compared to systems with shorter arms. The core, consisting of 0.003 inch thick spring steel shim stock embedded in mediprene elastomer, delivered a greater amount of force at more acute unfolding angles.

The milled steel center increased the unfolding impact force compared to a core made solely from mediprene. This effect was not seen if the mediprene material continued along the arm past the steel section. For example, in a 15 mm long mediprene core with a 7 mm long steel section, there existed no significant change in impact force between a device with and without the steel part (FIG. 8F). In an 8 mm long core, adding the 0.003 in thick steel resulted in a 60% increase in impact force. Adding the steel core also increased the force required for a 45° deflection and 180° torsion by 150% and 50% respectively (FIGS. 8G-3H). Steel pieces thinner than 0.003 inches commonly broke after multiple tests and those thicker commonly ruptured the mediprene coating.

In vivo, it was noted that devices with longer arms opened in the axial direction more frequently than devices with shorter arms (FIG. 8I). Axially oriented devices stretched the tissue to a diameter of 40 mm at its narrowest point in the plane perpendicular to the tissue's central axis. Devices which opened in the parallel direction with and without a metal core stretched the tissue to 12 and 10 mm respectively (FIG. 8J). Adding a metal core also increased the percentage of axial deployments. Without wishing to be bound by theory, the increased percentage of axial deployments may be due to the increased force for deflection generally limiting the device's flexibility to change conformations.

The actuating component fit inside of a custom designed capsule with a 9 mm diameter and 26 mm length (FIG. 8E). In some designs, the capsule possessed two chambers. The top one was waterproof and contained the actuating component while the bottom one possessed a moisture activated actuation mechanism. After entering the duodenum, a Eudragit L-100/55 shell dissolved and exposed two holes on the bottom of the capsule. Inside of the capsule's lower chamber, a polyethylene glycol (PEG) coating began to dissolve which was encasing two compressed springs in series. Once dissolved, the springs propelled the actuating component out of the capsule which unfolded and delivered the microneedle patches to the intestinal wall. The elastomeric core of the actuating component exited the capsule last, protecting the microneedles from shearing or compressing against the capsule structure. X-ray imaging confirmed capsule actuation occurred within two hours in vivo (FIG. 8C). In vitro, it was demonstrated that different molecular weight PEG coatings allowed the capsule to actuate and unfold over different time scales from 2-5 hours (FIG. 8L).

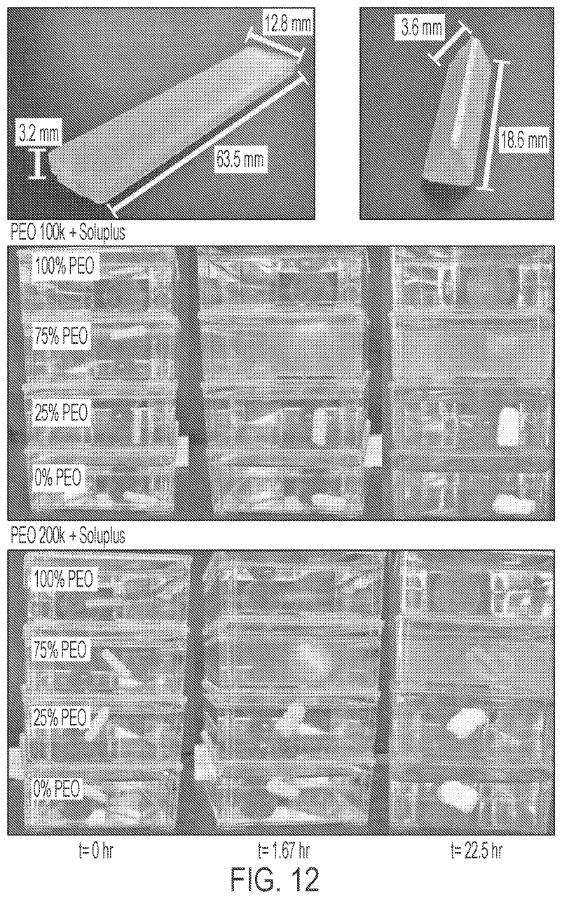

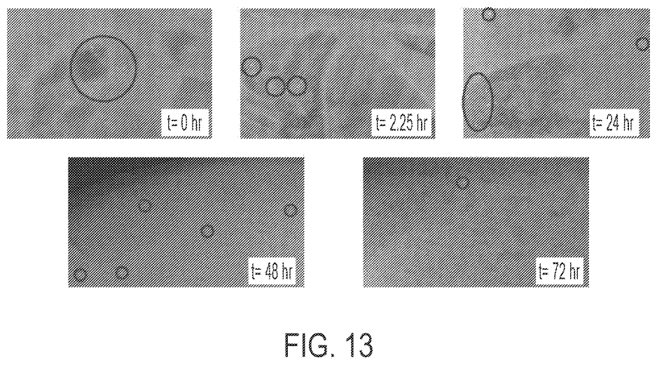

The unfolding arms were designed to ensure they maintained enough strength to deliver the drug payload in vivo while still dissolving in a timely manner to prevent obstruction. Fabricated from mixtures of biodegradable polyethylene oxide (PEO) and Soluplus®, the arms completely dissolved within 24 hours in vitro and in vivo (FIGS. 12-13). Still, the material retained enough mechanical strength during dissolution to push against the tissue wall for 10 minutes as determined via a 3 point bend test (FIG. 8M). As the arms dissolved, a Eudragit coating holding the capsule together also degraded and allowed the capsule to break into two pieces, each 9 mm in diameter and 15 mm in length. The non-degradable elastomeric core of the actuating component, measuring 12 mm in diameter and 1.5 mm in height, passed through the GI tract along with the capsule parts without issue during all in vivo experiments.

### Example 3—Microneedle Delivery

The following example demonstrates the characterization of the penetration of tissue by the microneedle patch of the actuating component such as those described in Example 1.

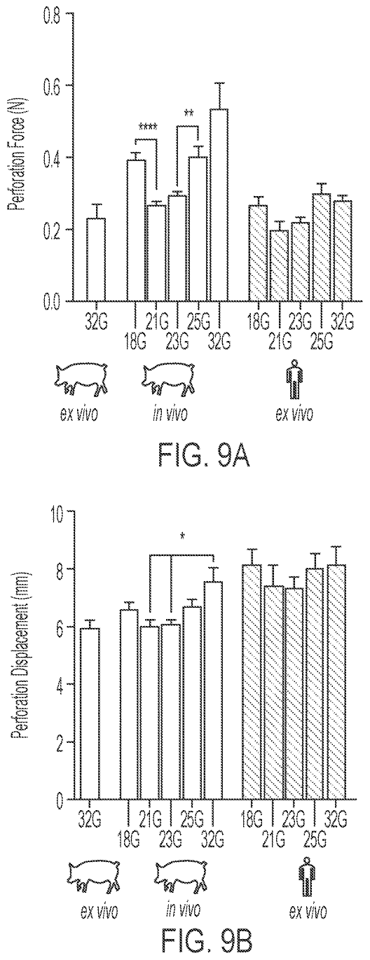

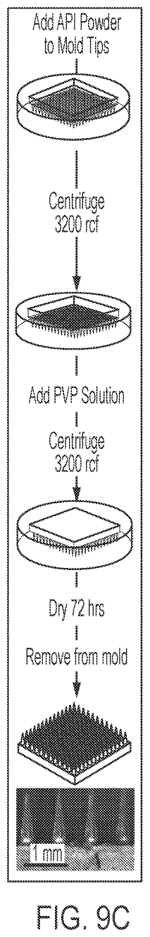

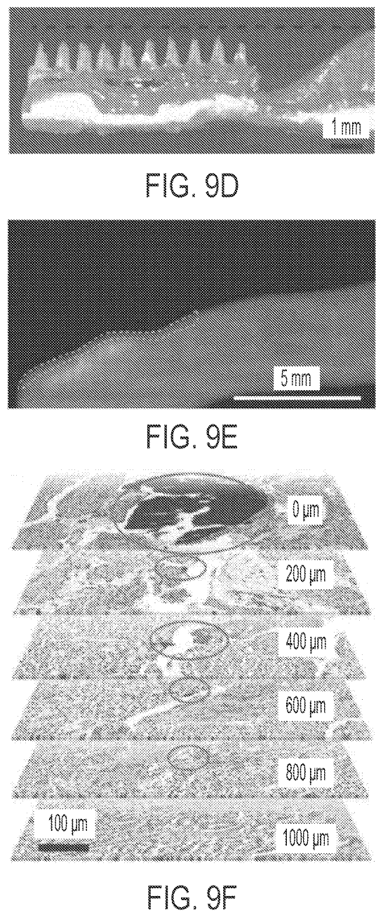

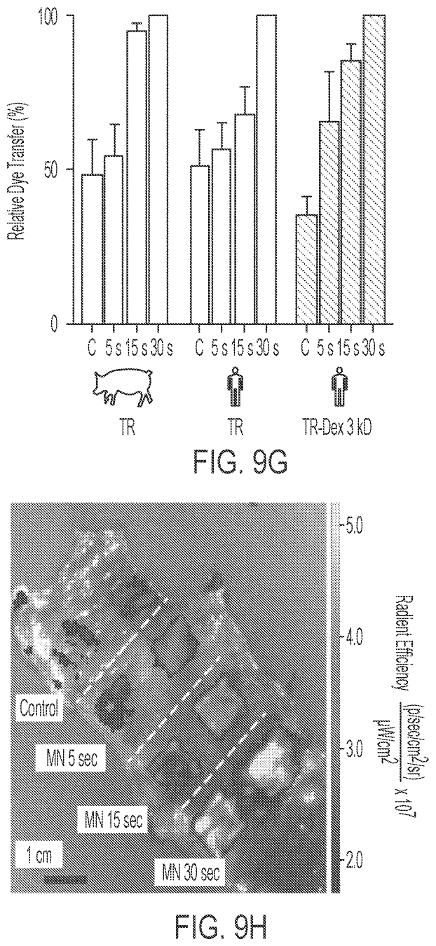

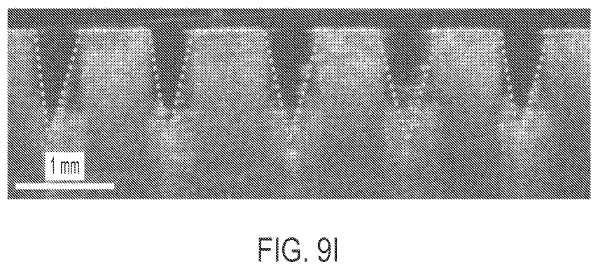

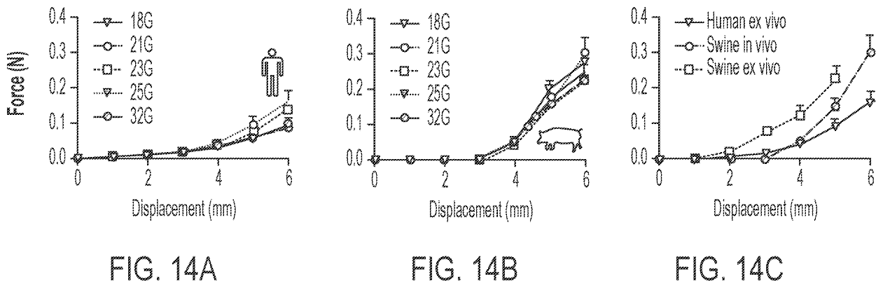

Penetration experiments were performed on ex vivo human small intestine tissue as well as on ex vivo and in vivo swine tissue to determine the force and distance required to generate a full thickness perforation. Camera footage showed the needle entered the tissue after applying as low as 5 mN of force. The needle reached a displacement of over 6 mm before perforating the outermost tissue layer. Hypodermic needles of all sizes required similar amounts of force to displace the tissue 6 mm (FIG. 14). Ex vivo human tissue required less force for tissue displacement compared to both in vivo and ex vivo swine tissue. Perforation forces for in vivo swine tissue ranged from 0.27 N-0.53 N, compared to 0.20 N-0.28 N for ex vivo human and swine tissue perforation (FIG. 9A). Depending on the needle gauge size, ex vivo and in vivo swine small intestine tissue perforated after 6-8 mm and ex vivo human tissue after 7-8 mm of tissue displacement (FIG. 9B). Thin needles, such as 32 G needles, generally used both a greater displacement and force for tissue perforation compared to 21 G and 23 G needles during in vivo experiments. This may have been due to shaft buckling and tip hooking from tissue movement as the swine breathed. Using the results from these experiments the exemplary system was designed with an arm impact force measuring 0.41±0.06 N, which delivered a low enough force to avoid perforation. After deciding on the device specifications, a microneedle patch platform was designed for the actuating component to deliver high loads of API.

A novel method for microneedle fabrication utilizing API powder was developed in order to increase the drug loading for the actuating component (FIG. 9C) and incorporated an outward facing indentation in the actuating component arms to accommodate the microneedle patches (FIG. 9D). The elastomer core placed stress on the arms during encapsulation, and the indentation ensured that the microneedle tips did not break against the capsule wall, maintaining their sharpness to penetrate the tissue. Each actuating component held one microneedle patch on each arm and possessed a total microneedle cross sectional area of 0.5 cm². This allowed the exemplary actuating component to hold up to 0.3 mg of drug.

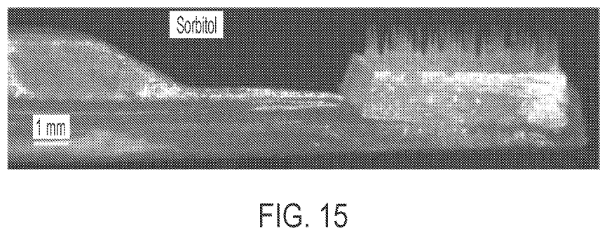

The actuating component was also able to load multiple formulations and active pharmaceutical ingredients by incorporating microneedle patches made with insulin, lysozyme and alpha-glucosidase onto the actuating component (FIG. 15). These included patches which used either polyvinylpyrrolidone or sorbitol as a binding ingredient.

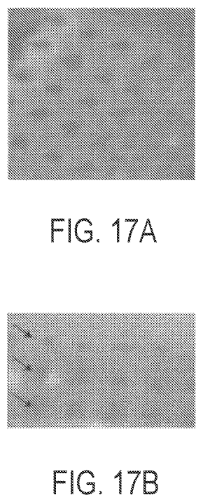

Multiple imaging techniques confirmed microneedle penetration in ex vivo small intestine tissue. MicroCT pictures of actuating components loaded with barium sulfate microneedle patches (FIG. 9E) and hypodermic needles (FIGS. 16A-16C) demonstrated tissue penetration in ex vivo small intestine without perforation as denoted by the pink dotted lines. These results were further supported using histology where hypodermic needles coated with tissue marking dye penetrated 800 μm through the tissue (FIG. 9F). The absence of dye in the 1 mm slice suggested that the needle did not reach this depth, reassuring its inability of perforating the small intestine. Optical coherence tomography (OCT) also confirmed microneedle penetration after the deployment of a single actuating component arm into the tissue from a 30 degree angle (FIGS. 17A-17B).

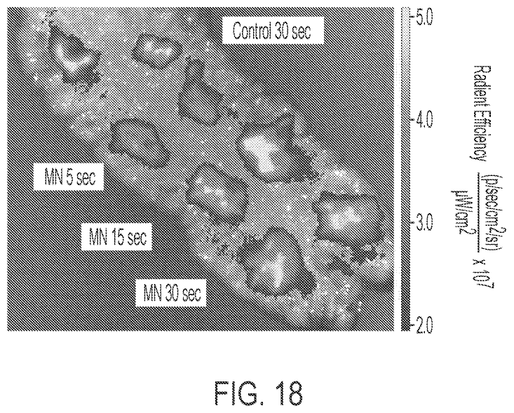

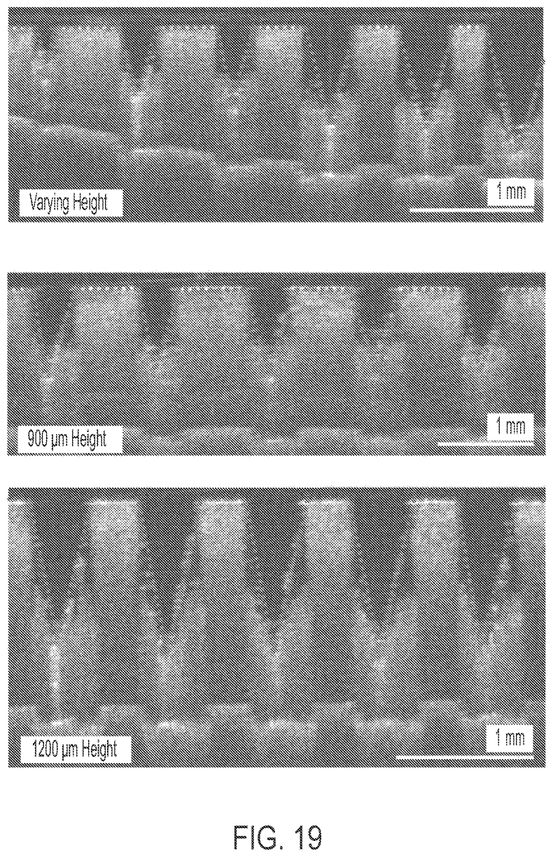

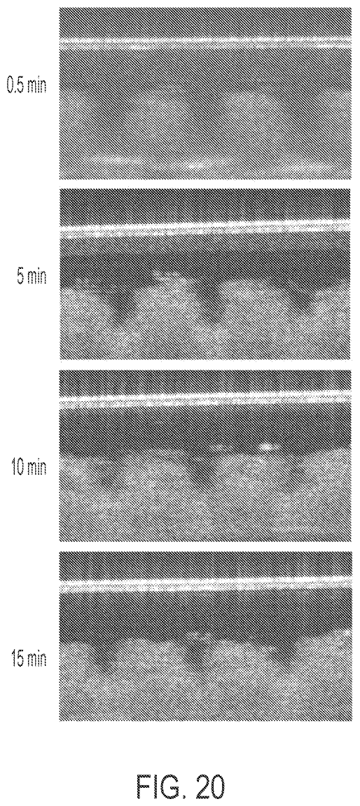

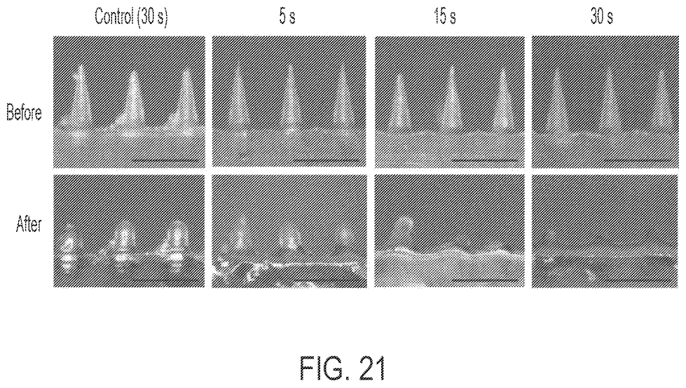

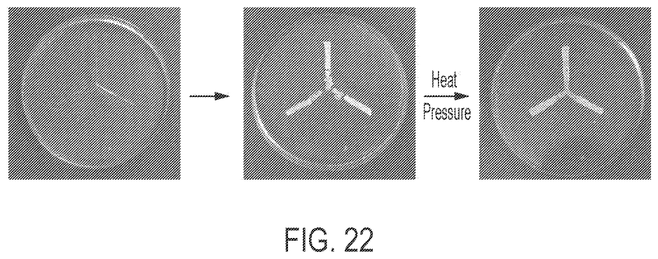

Microneedle dissolution patterns were studied and, in turn, drug delivery kinetics using both insulin and Texas red-based fluorescent dyes. Up through 30 seconds, increasing residence time correlated with increased levels of dye transfer in both ex vivo human and in vivo swine tissue (FIGS. 9G-9H, FIG. 18). A microneedle patch was rested on top of the tissue without any insertion force to act as a negative control, to determine dye deposition due to contact as opposed to penetration. Penetration and dissolution events were further confirmed using OCT and optical microscopy (FIG. 9I and FIGS. 19-21). Through these studies it was confirmed that microneedles successfully penetrated into small intestine tissue, rapidly dissolved upon insertion and left their payload inside of the tissue.

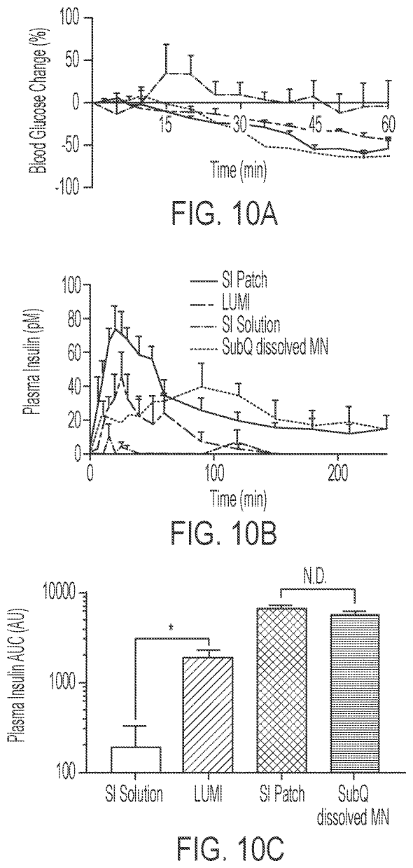

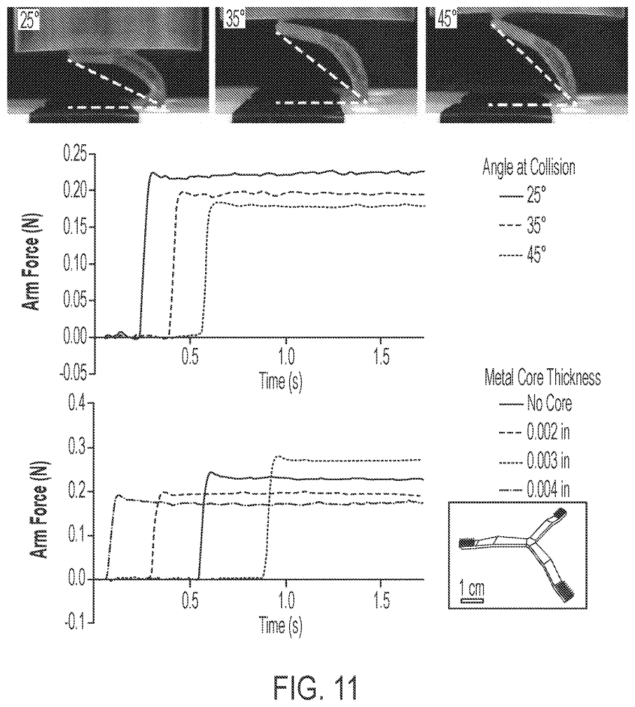

When released in the small intestine in vivo, actuating components loaded with insulin delivered drug systemically and achieved a peak plasma concentration comparable to subcutaneous dosing. In total we delivered 0.6 mg of drug and 1 cm² of microneedles in each experiment. In one set of experiments, we placed and released two actuating components per swine in the jejunum. This method of delivery provided a 44%±5% blood glucose drop over 60 minutes (FIG. 10A). Comparatively, subcutaneous dosing of a 1 cm² microneedle patch dissolved in 0.5 mL of sterile saline produced a 64%±12% blood glucose drop. Direct microneedle patch application to the small intestine tissue yielded a 54%±8% blood glucose drop. Dissolved microneedles delivered to the small intestine in a 10 mL solution showed no significant blood glucose level changes. We recorded blood glucose level changes over 4 hours, but several of the swine required dextrose infusions after the first hour to rescue them from hypoglycemia. Actuating component dosed swine possessed peak serum insulin levels of 46 pM±15 pM, and insulin remained in the blood for the entire four hour monitoring period (FIG. 10B). Subcutaneously dosed swine saw a peak systemic insulin concentration of 39 pM±15 pM.

Over the course of 4 hours, the microneedle patch applied to the small intestine and the subcutaneously dosed insulin delivered an equivalent systemic drug uptake (FIG. 10C). The actuating component systemically delivered 33% of the subcutaneous dose. The actuating component and the small intestine microneedle patches reached peak systemic insulin concentrations 25 min after dosing compared to 90 min for the subcutaneous administration.

### Example 4—Safety and Capsule

The following example demonstrates the safety and delivery design of exemplary actuating components, such as those described in Examples 1 and 2.

The experiments performed addressed the safety and efficacy of microneedle penetration in the GI tract. The tissue penetration tests performed with human and swine tissue, in combination with the “bed of needles” effect, reinforced the notion that the actuating component provided no significant risk for microneedle perforation. A comparative device containing 30 microneedles would generally require on the order of 3 N to perforate the tissue with each needle. The actuating component described in Examples 1 and 2 delivered a force on the order of 10 times less than comparative devices. Still, it was possible that a device would deliver an array of needles such that the force was unevenly distributed. In fact, literature on transdermal microneedle patch delivery demonstrated that applicators applied a disproportionate amount of force to microneedles on the corners of patches. The small intestine in particular possessed an uneven surface due to folds and villi projections, which made the tissue more susceptible to uneven force distributions. The experiments described herein showed through histology and microCT that no perforation event occurred even with an unfolding actuating component containing a single hypodermic needle on each arm.

The risk of small intestine obstruction, a medical condition sometimes requiring hospitalization and surgery, generally increases with the presence of large non-degradable objects. The actuating components described in Examples 1 and 2 were designed to dissolve and break apart into small pieces to avoid this complication. The Pillcam™, an ingestible non-dissolving capsule endoscopy system, measures 11 mm in diameter and 26 mm in length. During a study, these capsules retained for greater than 24 hours within the GI tract at a rate of 1.4%. Case reports have demonstrated that Pillcam™ retention sometimes led to GI obstruction. While this obstruction and retention rate was acceptable for devices dosed once every several years, daily dosed devices require more stringent safety limits. Many ingestible and non-degradable devices in preclinical development exhibit dimensions similar to the Pillcam™. Obstruction risk may prevent these larger devices from passing clinical trials.

An exemplary actuating component utilized the OROS osmotic pump capsule, a daily dosed and non-degrading drug delivery device, as a model for device size. One version of the OROS measured 12 mm in diameter and 5 mm in thickness with an obstruction rate of less than 1 in 50 million during commercialization. Another version of the OROS measured 9 mm in diameter and 15 mm in length. with a gastric retention rate of only 1 in 22 million. During our experiments, the actuating component left behind non-degradable pieces equivalent in size to the OROS system. After the arms degraded, the actuating component 1.5 mm thick and 12 mm in diameter core possessed dimensions smaller than the OROS pill. The capsule also broke up into smaller pieces (9 mm in diameter and 15 mm in length), comparable in size to the second OROS system. Therefore, it is expected that the rates of gastric obstruction would remain inconsequential during further translation efforts.

While certain sensations such as pain from injection do not exist in the GI tract, discomfort arises when the small intestine bloats and stretches. Because the actuating component functioned by stretching the small intestine to inject needles, discomfort may have arisen during device actuation. These feelings were not observed as deployment occurred while pigs were sedated. Pigs were monitored daily after delivering the devices, and they showed no signs of discomfort. The fast dissolution time for the arms on the order of a few hours ensured that the stretch would only occur for as long as necessary to deliver the drug payload. No changes in behavior or eating habits were observed until capsule excretion.

The exemplary actuating components generally used gastric emptying to move from the stomach to the small intestine. Gastric emptying times vary significantly between people. Emptying typically occurs in 1-4 hours, but individuals experiencing gastroparesis—common in diabetic patients-may face gastric emptying times as long as 24 hours. Ultimately, the actuating component provided a safe and effective platform technology for injecting microneedles into small intestinal tissue. It effectively delivered insulin systemically in a swine model. The actuating component could potentially deliver any drug formulation mentioned in the microneedle literature including vaccines, monoclonal antibodies, enzymes, hormones, and many other compounds which currently lack oral formulations. Clinical translation of orally delivered GI microneedle injections could lead to a paradigm shift in the delivery of macromolecules.

### Example 5—Materials, Testing, and Fabrication

The following example demonstrates exemplary materials and fabrication methods that may be used to fabricate actuating components, such as those described in Examples 1-3.

### Materials

Dulbecco's Phosphate-Buffered Saline (PBS) was purchased from Gibco by Life Technologies (Woburn, USA). Human insulin was obtained from Novo Nordisk (Maalov, Denmark). Soluplus® (polyvinyl caprolactam-polyvinyl acetate-polyethylene glycol graft copolymer (PCL-PVAc-PEG)) was purchased from BASF (Ludwigshafen, Germany). The 100,000 and 200,000 molecular weight poly(ethylene oxide) (PEO), along with the sulforhodamine 101 acid chloride (Texas red) was purchased from Sigma Aldrich (Natick, USA). Polyvinylpyrrolidone, average M.W. 58,000, was obtained from Alfa Aesar (Haverhill, USA).

Polydimethylsiloxane (PDMS) Sylgard 184 was purchased from Dow Corning (Midland, USA). Female Yorkshire swine were obtained from Tufts University (Medford, USA) and excised swine tissue from the Blood Farm Slaughterhouse (West Groton, USA). Human tissue was provided within 24 h of retrieval by the National Disease Research Interchange (NDRI, Philadelphia, USA). The Blue CDI's Tissue Marking Dye® was purchased from Cancer Diagnostics (Durham, USA). Mediprene 4410-LP11L was obtained from Lubrizol (Wickliffe, USA). Eudragit L 100-55 and Eudragit S100 were obtained from Evonik (Essen, Germany). 316 stainless steel shim stock was obtained from McMaster Carr (Elmhurst, USA).

### Actuating Component Fabrication

Three dimensional actuating component models were designed in Solidworks (Dassault Systemes, Velizy-Villacoublay, France) and printed out on an Objet 30 Pro 3D printer (Stratasys, Eden Prairie, USA). A negative mold was created out of PDMS. Stainless steel cores milled on an OtherMill V2 (Bantam Tools, Berkeley, Calif.) were encased in mediprene and added to the center core of the mold. A mixture of 25% Soluplus® and 75% PEO 200 kDa was microcompounded on an Xplore™ twin screw microcompounder (Xplore™ Instruments, Netherlands) at 50 rpm. This mixture was added to the arm sections of the mold. Using a Master-Mite model 10008 heat gun (McMaster Carr, Elmhurst, Ill.), the materials were melted. The metal core was aligned to the center of the device. Pressure was then applied to the mold and the materials were allowed to cool.

Fabricated microneedle patches were then placed on the recessed sections of the actuating component arms. The base plates of the patches were sanded down to a thickness of 1 mm and the patches were cut into 4×8 microneedle arrays of drug loaded microneedles. The arms of the device were then reheated using a heat gun, and the patches were placed into the melted sections of the arms.

### Actuating Component Tissue Penetration Characterization

Single hypodermic needle perforation testing in vivo was performed by affixing a needle to a 10 N Shimpo force gauge (Cedarhurst, USA). The force gauge was attached to an arm on a custom stage. A motor was used to move the arm downwards at a rate of 0.2 mm/s. A camera was placed on the moving stage to visualize the penetration event. The force measurements and video feed were recorded in LabVIEW (National Instruments, Austin, USA). Yorkshire swine were sedated as described in the in vivo section, and a laparotomy procedure was performed to access the small intestine. A 5 cm incision was made in the small intestine to reveal a working area of 5 cm by 1.5 cm, and the tissue was fixed so that it was held taut. The needle was then placed directly over the tissue and moved downward at the defined rate until we were able to visualize the needle on the other size of the tissue. All perforation events were correlated to a force drop. Breathing affected intraoperative measurements, and it was determined that the displacement caused by the breathing accounted for an extra 3 mm of penetration. This distance change was measured using a ruler and confirmed it by analyzing the force vs displacement curves. It was confirmed that forces during the exhaled state were equivalent to forces during the inhaled state 3 mm earlier.

Single hypodermic needle perforation testing ex vivo was performed using an Inston 5943 machine equipped with a 10 N load cell (Norwood, USA). Harvested tissue was affixed to a corkboard with a 2.5 cm diameter hole. Needles were fixed to the Instron machine's cross-head and lowered into the tissue above the hole at a rate of 0.1 mm/s until we visualized the needle on the other side of the tissue. All perforation events were correlated to a force drop.

Penetration of the microneedles attached to the actuating component were tested by performing histology and microCT on ex vivo swine tissue. MicroCT imaging was performed on a GE CT120 microCT imaging system (General Electric, Boston, USA). The devices were deployed with either sharpened metal hypodermic needles or with microneedles loaded with barium sulfate (Sigma Aldrich). The needles were also coated in a tissue marking dye (Cancer Diagnostics Inc, Durhan, USA) in order to mark the area of tissue penetration for histology.

### Actuating Component Arm Dissolution Characterization

Mixtures of either 100 kDa or 200 kDa PEO and Soluplus® were combined in an Xplore™ twin screw microcompounder (Xplore™ Instruments, Netherlands) at 50 rpm. The extruded material was captured in an Xplore™ 5.5 cm³ laboratory injection molding machine and molded in to an equilateral triangular prism with side lengths of 3.6 mm and a height of 18.55 mm. The machine exerted 3 bars of pressure for 1 second, ramped up to 4.5 bars over 1 second, and held a pressure of 4.5 bars for an additional 5 seconds.

Homemade Simulated Intestinal Fluid (SIF) was made by mixing 6.8 g of KH₂PO₄ Potassium phosphate monobasic (Sigma Aldrich) with 0.896 g NaOH (Sigma Aldrich) in 1 L of nanopure water. The pH was confirmed to be at 6.8 using the Mettler Toledo FiveGo pH meter (Columbus, USA).

Eight 250 mL Falcon tissue culture flasks (Corning, Corning, USA) were labeled and used to house each mixture. A volume of 225 mL of SIF was inserted into each flask and stored at 37° C. in an Innova 44 incubator (Eppendorf, Hamburg, Germany) which was being shaken at 50 rpm. The 50 rpm agitation simulated the intestinal environment. The flasks containing only SIF were left in the incubator for 6 hours to allow for temperature equilibration. One extruded shape was dropped inside each flask. The shapes were photographed at the 5 min, 1.67 h and 22.5 h time points and the appearance of the arms and the SIF were noted.

Additionally, the mechanical properties of the PEO and Soluplus® mixtures were determined during the dissolution process. Using the same microcompounding and extrusion method described above, bars of the mixtures measuring 3.2 mm×12.8 mm×63.5 mm were created. Three point bend tests were performed on the bars using a uniaxial mechanical tester (Instron 5943, Norwood, USA). These bend tests were performed on bars that were not placed in any liquid bath as well as bars that were placed in a shaken and incubated mixture of SIF for 10 minutes. Bars were fixed on a three point bend fixture (Instron) with support pins placed 36 mm apart. The cross-head was moved at a rate of 10 mm/min. Maximum flexural strength was calculated from the maximum load using the following equation:

$$σ = \frac{3 F L}{b {d}^{2}}$$

where F is the load at the fracture point, L is the length of the support span, b is the width of the bar, and d is the thickness of the bar. The maximum flexural strength for the actuating component arm was also calculated from this equation using the arm's dimensions.

### Capsule Fabrication

Three dimensional models of the capsule pieces were created in Solidworks and printed on an Objet 30 Pro 3D printer. The two body portions of the capsule were adhered together by spray coating Eudragit S onto the piece as they were clasped together. The bottom piece of the capsule was press fit into the bottom portion of the capsule's body. A spring with a compressed length of 4.114 mm, a load of 1.343 N, and a free length of 31.750 mm (Spring CI 011EF 11S, Lee Spring, Brooklyn, USA) was then trimmed to a length of 30 mm and cut in half. Using thread (Sparkfun, Niwon, USA), one half of the springs were tied to the bottom section of the capsule, and the other half were tied to the plunger. The two spring halves were then placed inside the capsule in series. Pressure was applied on the plunger to fully compress the spring. Melted PEG was then fed through the bottom of the capsule to freeze the spring in place. Molecular weights of PEG between 3,000 and 35,000 were used (Sigma Aldrich). The change in dissolution time allowed the capsule to release the device at different time intervals. The relationship between PEG molecular weight and capsule actuation was tested in a bath of SIF heated to 37° C. Eudragit L-100 55 was then spray coated onto the bottom of the device to coat the PEG. The actuating component was then placed inside of the capsule and the cap was pressed fit onto the top of the capsule.

### Microneedle Fabrication

Microneedle patches were fabricated with insulin concentrated in the tips. Solid insulin powder was placed in PDMS female microneedle molds and forced into the microneedle tips using a spatula. Excess powder was then removed from the mold. The amount of powder added to the mold was calculated by weighing the mold before and after the addition of powder. The accuracy of weight measurements was confirmed using high performance liquid chromatography. Briefly, a 7.8×300 mm² Insulin HMWP column (Waters Cerp, Milford, USA) was set to room temperature and an Agilent (Santa Clara, USA) HPLC machine was employed. Elution were performed at a flow rate of 0.5 mL/min for 26 minutes using a mobile phase made from 15% acetic acid (v/v), 20% acetonitrile (v/v), and 0.65 g/L L-arginine all purchased from Sigma Aldrich. The molds were then centrifuged at 3200 rcf for 10 minutes to compress the powder. Next, a 50% 58,000 molecular weight polyvinylpyrrolidone solution or 100% melted sorbitol was added to bind the powder and give mechanical structure to the microneedle patches. The mold was then centrifuged again at 3200 rcf for 10 minutes. The microneedle patches were left to dry at room temperature for 72 hours. Once dried, microneedles patches were unmolded, sanded down and mounted at the edges of the actuating component arms.

### Microneedle Dissolution

Microneedles loaded with Texas red and Texas red conjugated with dextran (3 kDa) were used to perform dissolution tests in vivo in swine prior to euthanasia and ex vivo in human small intestinal tissue. Microneedles were manually inserted for 5, 15 and 30 s and then retrieved. A microneedle patch was left to sit on top of the tissue without applying any pressure for 30 s which served as the negative control. An IVIS imaging system (Perkin Elmer, Waltham, USA) was then used to assess the Texas red and Texas red-dextran transfer onto the tissue via fluorescence. Living Image® software (Perkin Elmer, Waltham, USA) was used to quantify the radiant efficiency.

The dissolution experiment detailed above was also performed in vivo with insulin-loaded microneedles. The microneedles were imaged using an optical microscope before and after their application in the small intestine tissue to visually assess their dissolution.

### Microneedle Penetration

An optical coherence tomography (OCT) system was used to visualize penetration of the microneedles into excised small intestine from swine. For this, various microneedle arrays were inserted ex vivo into the tissue via manual application and OCT was used to evaluate both penetration depth and dissolution. In addition, a fixture was designed in order to hold the actuating component and deploy one of its arms in a 30 degree angle into a certain point of an ex vivo swine small intestine piece in order to promptly capture the penetration event prior to microneedle degradation. In this latter case, the OCT image was captured from the outside of the wall instead of from behind the patch, allowing in turn the assessment of perforation. OCT images were processed using Image J (Open Source).

### In Vivo Testing

To assess the insulin microneedle formulation, the API formulation was administered to female Yorkshire swine, 35 kg to 65 kg. To deliver the actuating components, the swine were placed on a liquid diet for 24 hours before the procedure and fasted the swine overnight, the swine were then sedated them with an intramuscular injection of Telazol (tiletamine/zolazepam) (5 mg/kg), xylazine (2 mg/kg), and atropine (0.05 mg/kg) and if needed supplemental isoflurane (1 to 3% in oxygen) via a face mask. An orogastric tube or overtube was placed with guidance of a gastric endoscope and remained in the esophagus to ease the passage of the device. The overtube was fed through the stomach and into the small intestine. Encapsulated actuating components were passed through the overtube and placed into the small intestine. Non-encapsulated actuating components were inserted and actuated during a laparotomy procedure in which a 3 cm incision was used to access the small intestinal mucosa. During laparotomy experiments, the size of the small intestine was standardized to 20 mm in diameter by applying a clamp to the tissue. The microneedles delivered manually to the small intestine were also inserted during a similar laparotomy procedure in which a 3 cm incision was used to access the small intestinal mucosa, and a microneedle patch was manually inserted into the intestinal surface epithelium. Patches with an area of 1 cm² were applied to the jejunum of the swine. Pressure was applied to the patch for 30 seconds, and then the patch was removed from the small intestine. To create the subcutaneous dose required for administration the microneedles from four patches were dissolved into 2 mL of sterile saline (Hospira, Lake Forest, USA). The mixture was then filtered through a 0.2 μm filter and 0.5 mL of the resulting solution was administered to each swine subcutaneously. Lastly, the insulin solution dosed to the jejunum was prepared by dissolving the microneedles from one patch into 10 mL of water purified using a Barnstead Nanopure system (ThermoFisher, Waltham, USA). The solution was then passed through an endoscope directly into the jejunum of the swine.

Blood samples were obtained via a central venous line at time points including but not limited to every 10 minutes for the first two hours and every 30 minutes for hours 2-4. Blood samples were tested for glucose levels using a OneTouch Ultra glucose monitor by LifeScan Inc. (Milpitas, USA). Collected plasma and blood was analyzed. Briefly, the homogenous bead assay employed two monoclonal antibodies against human insulin, creating an acceptor-bead, insulin, and donor-bead layering. This generally generated a signal which was proportional to the concentration of insulin. Additionally, blood was analyzed using ELISA. Both tests utilized antibodies specific for human insulin and neither test detected other endogenous insulins.

Specialized articles comprising the actuating components were administered to the swine to determine the capsule actuation time as well as the transit and dissolution timeline for the actuating component. These actuating components contained small pieces of metal material such as nitinol or stainless steel which allowed the device to be seen under X-ray. The swine were X-rayed over several hours in the case of the capsule actuation experiments. The swine were X-rayed over several days in the case of the transit experiments until the all of the metal components passed through the GI tract.

### Actuating Component Opening Geometric Analysis

A geometric analysis of the unfolding event defined a minimum arm length correlated with tissue stretch from any possible orientation. It was assumed that the small intestine possessed a known diameter (d) and the tissue was not rigid. The actuating component could open up in any orientation, including: axial; parallel; or anywhere in between. An analysis of all possible orientations showed that the tissue would stretch the least in the configuration where the planes perpendicular to the central axis containing an arm's point of contact were spaced furthest apart. Therefore, the arms contacted the tissue over the greatest possible surface area. In this orientation, we noticed that the small intestine conformed to the actuating component and changed shape. The tissue transformed from a cylinder and collapsed into two parallel rectangular sheets. Because the surface area of the small intestine remained constant, the height of this newly created rectangle equaled ½ of the small intestine's perimeter. The height of this triangle, created by the actuating component's three points of contact, corresponded to 1.5 times the actuating component arm length. Therefore the arm length was generally less than πd/3 in order to force the small intestine to stretch when the actuating component opened in this configuration, although other lengths are also possible.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Any terms as used herein related to shape, orientation, alignment, and/or geometric relationship of or between, for example, one or more articles, structures, forces, fields, flows, directions/trajectories, and/or subcomponents thereof and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, and/or geometric relationship include, but are not limited to terms descriptive of: shape—such as, round, square, circular/circle, rectangular/rectangle, triangular/triangle, cylindrical/cylinder, elliptical/ellipse, (n)polygonal/(n)polygon, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc.; direction—such as, north, south, east, west, etc.; surface and/or bulk material properties and/or spatial/temporal resolution and/or distribution—such as, smooth, reflective, transparent, clear, opaque, rigid, impermeable, uniform(ly), inert, non-wettable, insoluble, steady, invariant, constant, homogeneous, etc.; as well as many others that would be apparent to those skilled in the relevant arts. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more fabricated articles that would described herein as being “aligned” would not require such articles to have faces or sides that are perfectly aligned (indeed, such an article can only exist as a mathematical abstraction), but rather, the arrangement of such articles should be interpreted as approximating “aligned,” as defined mathematically, to an extent typically achievable and achieved for the recited fabrication technique as would be understood by those skilled in the art or as specifically described.

1. An article configured for administration to a subject, the article comprising:
a capsule having a body comprising
a first compartment and
a second compartment not in fluid communication with the first compartment, wherein both the first compartment and the second compartment, in a pre-deployment state of the article, are sealed from fluid communication with an environment external to the article;
a deployment mechanism associated with the first compartment and configured to eject, from the second compartment, a component for release internally of the subject;
a fluidic gate between the first compartment and an environment external to the first compartment, the fluidic gate having a first configuration in which the fluidic gate inhibits fluid communication between the external environment and the first compartment, and a second configuration in which the fluidic gate allows fluid communication between the external environment and the first compartment; and
a deployment inhibitor associated with the deployment mechanism, the deployment inhibitor configured to maintain the deployment mechanism in a pre-deployment state until sufficient exposure to a bodily fluid of the subject through a pathway including the fluidic gate in its second configuration,
wherein the deployment mechanism is configured to re-configure, in sufficient presence of the bodily fluid, allowing the deployment mechanism to eject the component from the article into an environment internally of the subject.

2. An article configured for administration to a subject, the article comprising:
a capsule having a body comprising a first compartment and a second compartment, the first compartment comprising a deployment mechanism associated with a fluidic gate embedded in a bottom portion of the first compartment;
a plunger disposed within the first compartment and associated with a tissue interfacing component disposed within the second compartment, wherein the plunger is configured to prevent fluidic communication between the first compartment and the second compartment; and
a deployment inhibitor associated with the deployment mechanism, the deployment inhibitor configured to maintain the deployment mechanism in a undeployed state until exposure to a fluid, wherein the deployment inhibitor is configured to disassociate in the presence of the fluid, actuating the deployment mechanism,
wherein the capsule body is sealed.

3. An article as in claim 1, wherein the first compartment and the second compartment are fluidically isolated.

4. An article as in claim 1, wherein the second configuration of the fluidic gate comprises a dissolution of at least a portion of the fluidic gate.

5. An article as in claim 1, wherein the deployment mechanism is impermeable to the bodily fluid.

6. An article as in claim 2, wherein the capsule maintains the tissue interfacing component in a dehydrated state until release.

7. An article as in claim 2, wherein the plunger is impermeable to the fluid.

8. An article as in claim 2, wherein the tissue interfacing component is a self-righting article.

9. An article as in claim 1, wherein the capsule body comprises a material non-dissolvable in the fluid.

10. An article as in claim 1, wherein the deployment mechanism is retained within the capsule body after release.

11. An article as in claim 1, wherein the capsule further comprises a cap associated with the second compartment.

12. An article as in claim 1, wherein the capsule has a largest cross-sectional dimension along a transverse axis of the capsule of less than or equal to 11 mm.

13. An article as in claim 1, wherein the capsule has a largest length of less than or equal to 26 mm.

14. An article as in claim 1, wherein the capsule further comprises a lubricant associated with the plunger.

15. An article as in claim 1, wherein the fluidic gate comprises a plug.

16. An article as in claim 15, wherein the fluidic gate comprises an enteric plug.

17. An article as in claim 1, wherein the capsule is configured to release the tissue interfacing component within 10 seconds of exposure of the deployment mechanism to the fluid.

18. A method for administering a tissue interfacing component to a subject, the method comprising:
administering, to the subject, a capsule having a body comprising a first compartment and a second compartment, a deployment mechanism comprising a deployment inhibitor within the first compartment;
exposing the capsule to a fluid having a pH of greater than or equal to 6 such that a fluidic gate having a first configuration and embedded in a bottom portion of the first compartment obtains a second configuration;
exposing the deployment inhibitor to the fluid such that the deployment inhibitor disassociates;
activating the deployment mechanism such that the deployment mechanism engages the tissue interfacing component disposed within the second compartment; and
releasing the tissue interfacing component from the capsule to a location internal to the subject.

19. A method as in claim 18, comprising releasing, from the article, an active pharmaceutical agent during and/or after releasing the tissue interfacing component from the capsule to a location internal to the subject.

20. A method as in claim 18, comprising orienting the article such that a longitudinal axis of the tissue interfacing component is orthogonal to the tissue located proximate to the article.
